(19) United States
(12) Patent Application Publication
(10) Pub. No.: US 2010/0240581 A1
(43) Pub. Date: Sep. 23, 2010

(54) SELECTIVE PROTEASOME INHIBITORS FOR TREATING DIABETES

(75) Inventors: Drew Tortoriello, Jersey City, NJ (US); Stuart P Weisberg, New York, NY (US)

Correspondence Address: LAW OFFICES OF ALBERT WAI-KIT CHAN, PLLC, 141-07 20th AVENUE, WORLD PLAZA, SUITE 604, WHITESTONE, NY, 11357, US

(73) Assignee: The Trustees of Columbia University in the City of New York, New York, NY (US)

(21) Appl. No.: 12/514,682

(22) PCT Filed: Nov. 13, 2007
(86) PCT No.: PCT/US07/23883

§ 371 Date: Oct. 30, 2009

Related U.S. Application Data
(60) Provisional application No. 60/858,838, filed on Nov. 13, 2006.

(51) Int. Cl. A61K 38/28; A61K 38/08; A61K 38/06; A61K 38/07; A61K 31/4015; A61K 31/353; A61K 31/7048; A61K 31/4995; A61K 38/16; A61K 31/12; A61K 31/195; A61K 31/407; A61K 31/18; A61K 38/10; A61K 31/192; A61P 5/50; A61P 3/10; A61P 29/00

(52) U.S. Cl. 514/6.7; 514/21.8; 514/21.9; 514/425; 514/456; 514/27; 514/250; 514/21.2; 514/679; 514/562; 514/421; 514/604; 514/21.5; 514/569

(57) ABSTRACT

Methods of modulating chronic low-grade inflammation are provided. More particularly, methods of treating diabetes, such as for example, type-2 diabetes mellitus, in a mammal by administering an effective amount of a selective proteasome inhibitor are provided. Also provided are unit dosage forms of such inhibitors.

### RELATED APPLICATION

This application relates to and claims priority to U.S. Provisional Patent Application No. 60/858,838, which was filed Nov. 13, 2006 and is incorporated herein by reference in its entirety.

### FIELD OF THE INVENTION

The present invention relates to methods for treating, preventing, and/or ameliorating the effects of diabetes, particularly type-2 diabetes mellitus, in a mammal. Such methods include administering to a mammal an effective amount of a selective proteasome inhibitor to treat, prevent, and/or ameliorate the effects of diabetes. The present invention also relates to methods of modulating chronic low-grade inflammation by administering selective proteasome inhibitors to a mammal. Unit dosage forms of such selective proteasome inhibitors are also provided.

### BACKGROUND OF THE INVENTION

Diabetes is a disease in which the body does not produce or respond to insulin, a pancreatic endocrine hormone crucial for cellular metabolism as well as for the prevention of hyperglycemia, a condition which over time fosters vascular disease leading to potentially devastating end-organ failure. Type-2 diabetes mellitus, which results from the body's inability to respond properly to the action of insulin, accounts for approximately 90% of all cases of diabetes worldwide.

Diabetes occurs most frequently in adults, but is being noted increasingly in adolescents, a finding attributable to the obesity epidemic (1,2). Although the plasma insulin concentration (both fasting and meal-stimulated) is usually increased, it is still insufficient for the degree of insulin resistance present and hyperglycemia results. With time, however, there is progressive β-cell failure and absolute insulin deficiency may ensue. In a minority of type-2 diabetic individuals, severe insulinopenia is present at the time of diagnosis and insulin sensitivity is normal or near normal.

Most individuals with type-2 diabetes exhibit visceral obesity, which is closely related to the presence of insulin resistance (4). In addition, these patients often have a clustering of abnormalities (hypertension, dyslipidemia, elevated PAI-1 levels) that confer upon them the diagnosis of the “metabolic syndrome” (5). Because of these abnormalities, patients with type-2 diabetes are at increased risk of developing macrovascular complications such as myocardial infarction or stroke.

Type-2 diabetes has a strong genetic predisposition and is more common in certain ethnic groups such as Mexican-Americans, Latinos, American Indians and Pacific Islanders (6, 7). The potentially subtle nature of its onset and symptoms renders nearly one-third of Americans with type-2 diabetes unaware of their afflicted status (8), an insidious situation given the fact that asymptomatic hyperglycemia can still provoke vascular disease and organ damage.

There are nearly 21 million Americans, or 7% of the population, who have diabetes. From a global perspective, the prevalence of diabetes is growing at an alarming rate. In 2000, the World Health Organization estimated that 177 million people world-wide had diabetes, and this number is predicted to double by the year 2025. The excess global mortality attributable to diabetes in the year 2000 was estimated to be 2.9 million deaths, equivalent to 5.2% of all deaths. Excess mortality attributable to diabetes accounted for 2-3% of deaths in the poorest countries and over 8% in the United States, Canada, and the Middle East. In people 35-64 years old, 6-27% of deaths were attributable to diabetes. Globally, diabetes is likely to be the fifth leading cause of death (9).

Because of its chronic, severe nature, diabetes is a costly disease from both a personal and federal level. Studies show that for low-income American families with a diabetic child, as much as 10% of family income may be devoted to diabetes care. In India, the corresponding figure would be 25%. In 2002, diabetes cost the United States an estimated $132 billion, of which approximately 70% was additional health care expenditures and 30% was lost productivity due to disability and early mortality (10). Diabetes increases the total health care costs of Americans up to three-fold.

Medical therapy of diabetes is limited to a few key drugs, which unfortunately are imperfect in their efficacies, side-effect profiles, and accessibilities. Given the staggering prevalence and expense of treating diabetes and its associated complications, it is imperative to investigate alternative and complementary treatments that would ideally be safe, effective, inexpensive, and readily available. There is, therefore, a need to develop new and efficient methods to prevent, treat, and/or ameliorate diabetes, particularly type-2 diabetes mellitus. The present invention is directed to meeting this and other needs.

### SUMMARY OF THE INVENTION

One embodiment of the present invention is a method for treating or preventing diabetes. This method comprises administering to a mammal an effective amount of a selective proteasome inhibitor to treat or prevent diabetes.

Another embodiment of the present invention is a method for treating or preventing type-2 diabetes mellitus. This method comprises administering to a mammal an effective amount of a selective proteasome inhibitor to treat or prevent type-2 diabetes mellitus.

Another embodiment of the present invention is a method of modulating chronic low-grade inflammation. This method comprises administering to a mammal in need thereof an effective amount of a selective proteasome inhibitor to modulate chronic low-grade inflammation.

A further embodiment of the present invention is a unit dosage form for treating or preventing type-2 diabetes mellitus. This unit dosage form comprises an effective amount of a selective proteasome inhibitor to treat or prevent type-2 diabetes mellitus in a mammal.

### BRIEF DESCRIPTION OF THE DRAWINGS

FIG. 1 is a diagram showing the organization and structure of the 26S proteasome. (A) Organization of the 20S catalytic core protease (CP). The position of the active-site threonines are shown. (B) Organization of the 19S regulatory particle (RP). (C) Diagram of the 26S proteasome combined with the predicted activities of the complex during degradation of ubiquitinated proteins. Adapted from: Vierstra, Trends Plant Sci., 8:55135-42 (2003).

FIG. 2 is a diagram showing that curcumin has pleiotropic effects, all of which are potentially beneficial for the treatment of diabetes or its complications.

FIG. 3 is a graph showing that dietary curcumin (3%) confers significant protective effect against hyperglycemia in male C57BL/6J ob/ob mice. N=5 per group; * signifies p<0.05 by two-tailed t-test.

FIG. 4 is a graph showing that dietary curcumin (3%) confers significant protection against hyperglycemia in male C57BL/Ks db/db mice. N=5 per group; * signifies p<0.05 by two-tailed t-test.

FIG. 5 is a graph showing that dietary curcumin (3%) decreases HbA1c percentage in all mouse models of diabetes tested. Non-diabetic mice were not affected. N=5-6 per group; * signifies p<0.05 by two-tailed t-test.

FIG. 6 is a graph showing that a single intraperitoneal injection of epoxomicin significantly lowers blood glucose for nearly 2 days thereafter in male C57BL/Ks db/db mice. N=5 per group; * signifies p<0.05 by two-tailed t-test.

FIG. 7 is a graph showing that a single intraperitoneal injection of celastrol significantly lowers blood glucose for nearly 2 days thereafter in male C57BL/Ks db/db mice. N=5 per group; * signifies p<0.05 by two-tailed t-test.

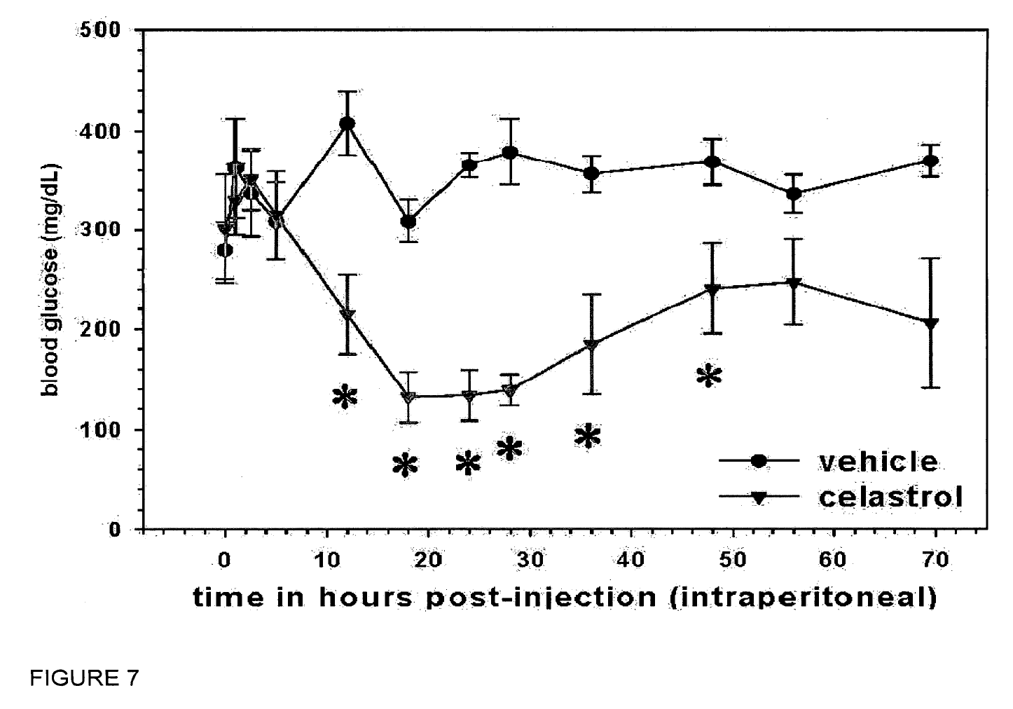

FIG. 8 is a graph showing that dietary curcumin significantly lowers AUC of glucose (A) but not insulin (B) tolerance test in male C57BL/6J DIO mice. n=5 per group; * signifies p<0.05.

FIG. 9 is a graph showing that dietary curcumin (3%) significantly lowers AUC of insulin tolerance test in male C57BL/6J ob/ob mice. n=5 per group; * signifies p<0.05.

FIG. 10 is a graph showing that celastrol injections significantly lower AUC of ITT in male C57BL/Ks db/db mice. n=5 per group; * signifies p<0.05.

FIG. 11 shows bar graphs summarizing Bruker NMR analyses of ob/ob mice treated with or without curcumin. In particular, NMR reveals that dietary curcumin (3%) significantly increases lean body mass and significantly decreases body weight and adipose mass in male C57BL/6J ob/ob mice. N=5 per group; * signifies p<0.05 by two-tailed t-test.

FIG. 12 shows bar graphs summarizing body fat percentages and liver weight in DIO mice treated with or without curcumin. In particular, one month of curcumin treatment is associated with significantly decreased body fat percentage and liver weight in male C57BL/6J DIO mice. N=6 per group; * signifies p<0.05 by two-tailed t-test.

FIG. 13 shows bar graphs summarizing Bruker NMR analyses of db/db mice treated with or without curcumin. In particular, NMR reveals that dietary curcumin (3%) is associated with significantly increased lean muscle mass and body weight in male C57BL/Ks db/db mice. An increase in liver weight was also noted. N=5 per group; * signifies p<0.05 by two-tailed t-test.

FIG. 14 shows that dietary curcumin significantly lowers hepatic expression of pro-inflammatory genes in male ob/ob mice after 10 weeks. (control values are set at 1); N=5 per group; * signifies p<0.05 by two-tailed t-test.

FIG. 15 shows that dietary curcumin significantly decreases hepatic NF-κB activity after 10 weeks. N=5 per group; * signifies p<0.05 by two-tailed t-test.

FIG. 16 shows that dietary curcumin significantly increases expression of adiponectin (Acdc) and decreases F480 (Emr1) expression in white adipose tissue of male ob/ob mice after 10 weeks. (control values are set at 1); N=5 per group; * signifies p<0.05 by two-tailed t-test.

FIG. 17 shows that dietary curcumin is associated with a significant reduction of adipose macrophage infiltration in male C57BL/6J ob/ob mice.

FIG. 18 shows that celastrol administered intraperitoneally for 3 days significantly increases expression of adiponectin (Acdc) while decreasing expression of Ccl2 (MCP-1) in white adipose tissue of male db/db mice. All values are mean±SEM; N=4-5 per group; * signifies p<0.05 by two-tailed t-test.

FIG. 19 shows that dietary curcumin significantly increased serum insulin levels in C57BL/6J db/db mice and decreased serum leptin levels in wild-type C57BL/Ks mice. N=5 per group; * p<0.05 by two-tailed t-test.

FIG. 20 shows the immunohistology of treated and untreated pancreatic islets from three different mouse models. By age 20 weeks, untreated C57BL/6J ob/ob mice (20A-C) and curcumin-treated C57BL/Ks db/db mice (20G-I) manifest hyperplasia of the pancreatic islets. Untreated C57BL/Ks db/db mice manifest islet depletion (20D-F). Arrows point to nuclei positive for Ks67, a proliferation marker.

FIG. 21 shows that intraperitoneal administration of a single dose of celastrol and epoxomicin significantly increase serum insulin in male C57BL/Ks db/db mice after 24 hours. N=6 per group; * signifies p<0.05 by two-tailed t-test.

FIG. 22 shows that PTEN and Foxo3a expression in pancreatic β-cells of male C57BL/Ks db/db mice is significantly decreased 24 hours after a single intraperitoneal injection of proteasome inhibitors. INGAP expression is significantly increased. All values are mean±SEM; N=6 per group; * signifies p<0.05 by two-tailed t-test.

FIG. 23 shows the effect of proteasome inhibition on the rat β-cell line INS-1. All proteasome inhibitors were able to significantly increase viable cell number compared to vehicle after 24 hours. However, at their highest concentrations, celastrol and epoxomicin exerted negative effects on cell viability.

FIG. 24 shows that proteasome inhibitors foster an increase in insulin secretion in Ins-1 cells after being in serum-free culture for 12 hours. The highest concentrations of epoxomicin exert a negative effect, likely due to cytotoxicity. All values are mean±SD; n=3 replicate wells per group.

### DETAILED DESCRIPTION OF THE INVENTION

It is believed that the present invention provides the first description of proteasome inhibition as a potent anti-diabetogenic agent in vivo. Indeed, the present invention is based on our discovery that inhibition of proteasomal activity reverses insulin resistance and prevents the inflammatory consequences of obesity by preventing, e.g., the degradation of insulin signaling molecules and IκB.

### The Proteasome: A Multimeric Proteolytic Tunnel

The balance between the rate of synthesis and degradation of any protein governs its relative cellular abundance and the time span of its activity. The half-life of such macromolecules can range from hours, in the case of gene products with housekeeping functions, to minutes for cell-cycle regulators, transcription factors, growth factors, or circadian regulators, which need to be active only transiently. A short half-life is also characteristic of either chemically or conformationally abnormal proteins. Unlike DNA, which is usually repaired when damaged by proof-reading DNA polymerases, damaged RNAs and proteins are quickly destroyed. Increasing their destruction rate is the fastest means of modulating their cellular levels and is generally achieved by increasing their accessibility or susceptibility to dismantling enzymes. Because proteases are compartmentalized within either lysosomal organelles or the macromolecular complexes known as proteasomes (11), proteolytic degradation is a restricted and highly regimented process.

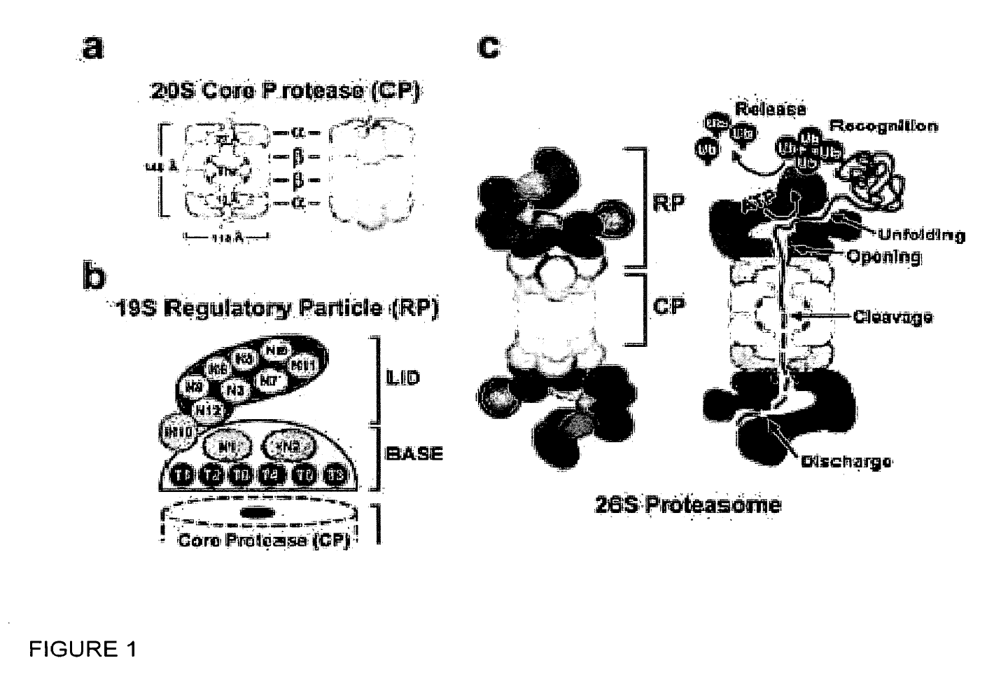

Proteasome-like proteins are present in all biological kingdoms and in most organisms. In the bacterium Escherichia coli, the HsIV protease forms two hexameric rings that pack like a “double donut”. A core “double donut” is also characteristic of the archaeal 20S proteasome, with 14 proteases (β subunits) arranged in two seven-membered rings (12) (FIG. 1A). The archaeal 20S proteasome has increased structural complexity compared to HsIV: the rings of β subunits are flanked on either side by an additional heptameric ring of α subunits. Both α and β subunits are structurally homologous to the HsIV protease, but only the β subunits are catalytically active. The eukaryotic 20S proteasome has a similar architecture with a stack of four seven-member rings, but exhibits greater complexity in terms of subunit composition as these rings are composed of seven different β subunits and seven different α subunits (13). Each β-ring contains three proteases, a chymotrypsin-like, a trypsin-like, and a post-glutamyl protease, for a total of six proteolytic active sites within the proteasome core. These multiple active sites are redundant to a certain extent as fewer active sites give similar proteolytic products. However, inhibition of the chymotrypsin-like activity is sufficient to block all catalytic activity of the proteasome. The four inactive β subunits are essential for the maintenance of the barrel-like architecture of the complex.

Eukaryotes have a targeting mechanism by which ubiquitin-tagged proteins are specifically recognized and degraded by the 26S proteasome. The 26S proteasome complex is composed of the 20S core and the 19S complex, which contains subunits of the AAA family of ATPases. The ring-like architecture of the proteasome features a hollow cavity with openings on both sides. In the proteolytic complex, the subunits are arranged such that the active sites of the β-subunits line the central chamber. Reconstruction by electron microscopy reveals that the 19S activating complex binds at the outer rims of the 20S core, where the entry pore lies (14). The 19s “cap”, complete with “lid” and “base” (FIG. 1B) restricts access to the central chamber via a narrow central pore, allowing only polypeptides in an extended conformation to be threaded towards the catalytic center. Consistent with the closed conformation of the entry pore observed in the absence of activators, the 20S proteasome purified from yeast displays very low in vitro activity, even toward unstructured substrates (11). Such a molecular architecture provides the basis for substrate selectivity in which only unfolded polypeptides but not folded domains are degraded by the proteasome. In addition, the 19s caps also serve as binding and deubiquitylation sites for ubiquitin-tagged proteins.

### The Ubiquitin/Proteasome Protein Degradation Pathway

The proteasome works in concert with a tagging protein, ubiquitin, to create the ubiquitin-proteasome pathway (UPP), the major proteolytic pathway of eukaryotes. Possible mechanisms of protein targeting to the UPP pathway likely include phosphorylation of a target protein by a signal transduction cascade, exposure of a hydrophobic protein surface via disaggregation of a protein complex or protein denaturation, specific N-terminal residues of the target protein and short amino acid sequences within the target protein. Once targeted, these proteins are covalently modified with polyubiquitin chains in a three-step, highly regulated enzymatic process involving a ubiquitin-activating enzyme (E1), ubiquitin-conjugating enzyme (E2), and ubiquitin ligase enzyme (E3).

The ubiquitin-activating enzyme (E1) is the first enzyme involved in the regulation of ubiquitylation. This enzyme uses energy derived from ATP to activate ubiquitin so that it can bind to proteins destined for degradation. However, before activated ubiquitin can be bound to a target protein it must be transferred from the E1 enzyme to one of 20 identified ubiquitin-conjugating enzymes (E2). The ubiquitin ligase or E3 enzyme recognizes and binds to specific target proteins and catalyses the transfer of activated ubiquitin from E2 to the target either directly or through a high-energy intermediate. By adding additional ubiquitin to lysine residues on the previously conjugated ubiquitin molecules, poly-ubiquitin chains are formed. Proteins marked with a polyubiquitin chain are delivered to a proteasome to be degraded. Targeted proteins are then recognized by the proteasome, unfolded, and degraded by the proteasome to peptides numbering 3 to 22 amino acids in length. Ubiquitylation can be considered a covalent post-translational modification and signal, comparable to acetylation, glycosylation, methylation, and phosphorylation.

### Current Clinical Application of Proteasome Inhibition:

The Velcade® Success Story

The ubiquitin-proteasome pathway plays an essential role in the degradation of proteins that are misfolded, oxidized, or damaged. The ubiquitin-proteasome pathway plays a key regulatory role in controlling the intracellular levels of a wide range of proteins, including those involved in the control of the cell cycle, transcriptional activation, apoptosis, and cell signaling. As such, proteasomes are key components of numerous biological pathways, including those related to the development of inflammatory and malignant disease. Therefore, manipulation of the proteasome's activity holds potential to interrupt the course of these disease processes.

In May 2003, the potential for proteasome inhibition to treat a clinical disease was realized when the U.S. Food and Drug Administration approved the reversible proteasome inhibitor bortezomib (PS-341, Velcade®) to treat patients with refractory multiple myeloma, a cancer of plasma cells. One cycle of Velcade® treatment entails the intravenous injection of 3.5 mg twice weekly for 2 weeks followed by a 10-day rest period (days 12-21). Cancer cells are killed while normal cells are spared. The mechanisms which have been postulated to underlie bortezomib's efficacy are several:
Velcade® blocks NF-kB activation in a dose- and time-dependent fashion by the inhibition of IκBα phosphorylation and degradation (15). Enhanced levels of IκBα lead to increased sequestration of the pro-inflammatory transcription factor NF-κB outside the nucleus. As a result, several NF-κB dependent genes that foster carcinogenesis, angiogenesis, metastasis, and severe inflammation are turned off. Although crucial, NF-κB inhibition is likely only one of many pro-inflammatory mechanisms potentially impeded by proteasome inhibition. In fact, one study using microarray analysis of RNA derived from murine macrophages treated with lipopolysaccharide in the absence or presence of the proteasome inhibitor lactacystin revealed that the vast majority of genes regulated by lipopolysaccharide are under the control of the proteasome (16). The products of these genes were determined to participate in no less than 14 distinct signaling pathways (11).
The failure to degrade cyclins inhibits completion of the cell cycle and hence the mitotic proliferation of the cancerous cells. The drug seems to work especially well when used with conventional chemotherapy, likely by inhibiting the ability of cancer cells to protect themselves against the damage chemotherapy induces.
Inhibition of Bcl-2 leads to death of the cell by apoptosis (17, 18).

The aforementioned beneficial effects induced by proteasomal inhibition would not likely be exclusively restricted to patients with multiple myeloma. In fact, proteasomal inhibition appears to have at least the potential to treat any disease with a significant inflammatory or hyper-proliferative aspect. Indeed, literature is rapidly accumulating, which delineates the salutary effects of Velcade® on several other diseases, many of which are non-malignant inflammatory conditions (19-22).

### Rationale and Potential Mechanisms Whereby Proteasome Inhibition can Improve Diabetes

Most individuals with type-2 diabetes are obese, a condition now perceived to be a state of chronic low-grade inflammation whose epicenter resides in white adipose tissue. Histologically, there exists an infiltration of macrophages into white adipose tissue that can be found encircling an increased number of dead adipocytes (23). Many inflammation-provoking “adipokines”, such as PAI-1 and MCP-1 (24, 25), and macrophage-specific genes, including TNF-α and IL-6, are significantly upregulated in the white adipose tissue of obese subjects. This upregulation precedes a dramatic increase in circulating insulin levels. Upon treatment with the insulin-sensitizer rosiglitazone, these genes normalize in expression.

Thus, obesity is a sub-clinical inflammatory condition in which the production of pro-inflammatory factors foster the pathogenesis of insulin resistance and diabetes (26, 27). The hyperglycemia of diabetes, if unchecked, fosters further inflammation via oxidative damage. This phenomenon, referred to as “glucose toxicity” (28), is believed to be responsible for the progressive β-cell failure noted in poorly controlled type-2 diabetics.

Inflammation is a major ontogenic factor in the development of both obesity and diabetes (29-33), and it is likely that the improved glucose tolerance induced by aspirin (34), adiponectin (35), thiazolidinediones (36), or statins (37) is related to their anti-inflammatory properties. It is therefore plausible that the anti-inflammatory effects of proteasome inhibition therapy would also favorably modulate the progression and course of diabetes. In addition, several molecules capable of delimiting the pathogenesis of diabetes have recently been identified as targets of the ubiquitin-proteasome pathway.

### IκBαNF-κB Blockade

Proteasome inhibition prevents NF-kB activation by inhibiting the degradation of its binding partner and inactivator, IκBα (15). As a result, several NF-κB dependent genes that foster severe inflammation are down regulated. In diabetic obesity, cytokines exert their pro-inflammatory effects predominantly through the NF-κB system. Genetic or pharmacological manipulation of this pathway is known to alter insulin sensitivity in animal models (38).

### Insulin Receptor Substrates: Insulin Signal Transduction

Insulin signaling has to be tightly controlled in magnitude and duration to maintain cell homeostasis. The protein amounts of the different insulin signaling molecules are regulated by their rates of synthesis and degradation. The ubiquitin-proteasome system is involved in the internalization of the insulin receptor, the regulation of transcription factors and nuclear receptors that mediate insulin-induced gene expression, the control of the amount of insulin receptor substrates (IRS) 1 and 2, and in the degradation of insulin itself.

Dysregulation of IRS-2 signaling in mice prevents the development of compensatory hyperinsulinemia during peripheral insulin resistance. IRS protein signaling is inhibited by serine phosphorylation or proteasome-mediated degradation, which might be an important mechanism of insulin resistance during acute injury and infection, or chronic stress associated with aging or obesity. Inflammation induces the expression of SOCS proteins, which bind IRS-1 and IRS-2, promoting their ubiquitylation and subsequent proteasomal degradation (39). Thus, SOCS-mediated degradation of IRS proteins, presumably via the elongin BC ubiquitin-ligase, might be a general mechanism of inflammation-induced insulin resistance, providing a target for therapy.

Regulation of IRS-2 expression is also critical to pancreatic islet β-cell survival. In the rat pancreatic β-cell line INS-1, chronic activation of the mammalian target of rapamycin (mTOR) by glucose and/or IGF-1 in β-cells leads to increased phosphorylation of IRS-2, a state which targets it for proteasomal degradation, resulting in decreased IRS-2 expression and increased β-cell apoptosis (40, 41). This may be a contributing mechanism as to how β-cell mass is decreased by chronic hyperglycemia in the pathogenesis of type-2 diabetes.

### MafA: Pancreatic β-Cell Survival

Chronic hyperglycemia in pancreatic β-cells greatly diminishes insulin gene expression, content, and secretion due to the loss of binding of transcription factors, most notably PDX-1 and MafA, to the insulin gene promoter region. Inflammation is a major ontogenic factor in the development of both obesity and diabetes (42, 43). Glucotoxic HIT-T15 β-cells possess normal amounts of MafA mRNA, but a severe reduction in MafA protein (43). Treatment of these cells with lactacystin, an irreversible proteasome inhibitor, caused an accumulation of MafA protein and corrected many of the negative effects exerted by “glucose toxicity” (43).

### KATP Channels

The β-cell KATP channel is a massive hetero-octameric complex of two types of protein subunits: four subunits of the inward rectifier potassium channel Kir6.2 and four subunits of the sulfonylurea receptor (SUR1) (44). KATP channels couple cell metabolism to electrical activity by regulating K⁺ flux across the plasma membrane. As a result, KATP channels exert a significant degree of control upon pancreatic β-cell insulin secretion. The number of active channels on the plasma membrane and their appropriate regulation are critical for proper β-cell function. Diseases such as familial hyperinsulinism and some forms of diabetes are directly attributable to KATP channel subunit mutations that result in aberrant trafficking and/or channel regulation (45-51).

The ubiquitin-proteasome pathway plays a key role in the biogenesis and surface expression of β-cell KATP channels. Both SUR1 and Kir6.2 subunits of the KATP channel are degraded by way of the ubiquitin-proteasome pathway (52). Interestingly, proteasomal subunit degradation occurs simultaneously, and with apparently similar rates, as does receptor assembly and trafficking (52). Thus, as subunits are synthesized, they are concurrently degraded, with both misfolded subunits, as well as functional assembly-competent subunits becoming degraded before they have the opportunity to assemble into a stable complex that is able to exit the ER. Therefore, proteasomal inhibition has the potential to increase insulin sensitivity by increasing the presence of β-cell surface KATP channels.

In the present invention, three selective proteasome inhibitors—curcumin, epoxomicin, and celastrol—are shown to reverse type-2 diabetes in animal models. Thus, we believe that selective proteasome inhibitors hold great promise for the sole or adjunctive treatment of diabetes, particularly, type-2 diabetes mellitus and quite possibly the metabolic syndrome in general.

As used herein, a “selective proteasome inhibitor” is a material, including natural extracts and synthetically derived compounds, which selectively prevents the degradation of intermediates in the insulin pathway, including for example, insulin signaling molecules and IκB, without adversely affecting proteasomal activities required for normal cellular function. In the present invention, members of the following classes of proteasome inhibitors may be used: (1) inhibitors of proteasome caspase-like activity, (2) inhibitors of proteasome trypsin-like activity, (3) inhibitors of proteasome chymotrypsin-like activity, and (4) inhibitors of all proteasome activities.

Inhibitors of proteasome caspase-like activity include for example, Ac-Ala-Pro-Nle-Asp-H, YU102, Calpain Inhibitor I (ALLN), ALLM (Calpain Inhibitor), Z-Ile-Glu(OBut)-Ala-Leu-H(PSI), MG115 (Z-Leu-Leu-Nva-H), MG-132 (Z-Leu-Leu-Leu-H), MG-262 (Z-Leu-Leu-Leu-B(OH)₂), Z-(Leu)₃-vinyl sulfone, and Z-Pro-Nle-Asp-H. Inhibitors of proteasome trypsin-like activity include, for example, lactacystin, clasto-lactacystin β-lactone, NIP-(Leu)3-vinyl sulfone, and TLCK. Inhibitors of proteasome chymotrypsin-like activity include, for example, aclacinomycin A (Aclarubicin), calpain inhibitor I (ALLN), ALLM (Calpain Inhibitor), epigallocatechin gallate, epoxomicin, gliotoxin, lactacystin, clasto-lactacystin β-lactone, NIP-(Leu)3-vinyl sulfone, phepropeptin A, phepropeptin B, phepropeptin C, Phepropeptin D, phepropeptin A, B, C, D Inhibitor Pack, TPCK, Z-Ile-Glu(OBut)-Ala-Leu-H(PSI), Z-(Leu)₃-vinyl sulfone, MG115 (Z-Leu-Leu-Nva-H), MG-132 (Z-Leu-Leu-Leu-H), MG-262 (Z-Leu-Leu-Leu-B(OH)2), and Z-Leu-Leu-Tyr-COCHO. Inhibitors of all proteasome activities include, for example, ada-(Ahx)3-(Leu)3-vinyl sulfone, ada-Lys(biotinyl)-(Ahx)3-(Leu)3-vinyl sulfone, ada-Tyr-(Ahx)3-(Leu)3-vinyl sulfone, bactenecin 5 precursor peptide (Bac5-GRR), PR11, PR26, and PR39. Additional non-limiting examples of proteasome inhibitors according to the present invention include ubiquitin+1 (Ub+1) and ubiquitin5+1 (Ub5+1). Preferably, the selective proteasome inhibitors according to the present invention are curcumin, epoxomicin, and celastrol.

In the present invention, derivatives of any of the foregoing proteasome inhibitors are contemplated. As used herein, “derivatives” of the selective proteasome inhibitors include enantiomers, optical isomers, diastereomers, N-oxides, crystalline forms, hydrates, and/or pharmaceutically acceptable salts thereof. The term “derivatives” also includes structurally similar compounds or extracts having the same or similar function of one of the proteasome inhibitors of the present invention. Moreover, the present invention includes the use of any combination of the foregoing proteasome inhibitors.

The natural compound, curcumin, is a polyphenol derived from the spice turmeric. The structure of curcumin is:

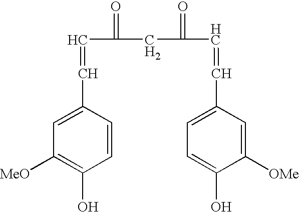

The dried ground rhizome of the perennial herb turmeric (Curcuma longa) has been used in Asian cooking and medicine (Ayurveda) for four thousand years. Its appeal is global—according to the Food and Agriculture Organization of the United Nations, over 2400 metric tons of turmeric are imported annually into the United States for consumer use. The polyphenolic phytochemical curcumin (diferuloylmethane) comprises 2-8% of most turmeric preparations and has potent anti-oxidant, anti-inflammatory, and anti-carcinogenic properties (53). It is currently the subject of several NIH sponsored chemoprevention trials.

Commercial grade curcumin is readily available in any health food store in the United States and is generally sold in capsules as a standardized 95% pure curcuminoid preparation with general recommendations to consume one or two 500 mg capsule three times per day to improve general well-being. It contains the curcuminoids curcumin (˜80%), desmethoxycurcumin (˜10-20%), and bisdesmethoxycurcumin (<5%). Studies in preclinical models of carcinogenesis have demonstrated that commercial grade curcumin has the same inhibitory effects as pure curcumin (54, 55).

Efficient first-pass effects and some degree of intestinal glucuronidation and sulfation limit oral curcumin's systemic availability. However, oral curcumin is detectable in the urine at relatively low oral doses in humans (56), suggesting that a significant amount of curcumin must enter the peripheral circulation. Perhaps more importantly, oral curcumin has been shown to exert beneficial effects within the body distal to the gastrointestinal tract (57, 58) without overt toxicity. Moreover, some studies have suggested that curcumin can elicit systemic effects, such as breast and liver chemoprevention, at tissue levels only in the nanomolar range (59, 60). This suggests that curcumin can exert potent systemic effects at very low plasma concentrations.

A number of murine preclinical studies, some of which were conducted for as long as 15 months and utilized exceedingly high dosages (2 g/kg/day), confirm curcumin's safety as an oral agent (61-63). Human studies are more limited, but none have reported any discernible toxicity. Administration of 1.2-2.1 g of oral curcumin daily to patients with rheumatoid arthritis in India for 2-6 weeks did not result in any reported adverse effects (64). In a study of high dose oral curcumin in Taiwan, Cheng and colleagues administered up to 8 g daily of curcumin for 3 months to patients with pre-invasive malignant or high risk pre-malignant conditions, stating that no toxicity was observed (57). In patients with advanced colorectal cancer treated in the UK, curcumin was well tolerated at doses up to 3.6 g daily for up to 4 months (56). In this study, one patient consuming 0.45 g daily and one patient consuming 3.6 g daily developed diarrhea (US National Cancer Institute (NCI) grades 1 or 2) one month and four months into treatment, respectively. One patient consuming 0.9 g curcumin daily experienced nausea (NCI toxicity grade 2) which resolved spontaneously despite continuation of treatment. Two abnormalities were detected in blood tests: a rise in serum alkaline phosphatase level was observed in four patients, consistent with NCI grade 1 toxicity in two patients and grade 2 toxicity in two patients; serum lactate dehydrogenase rose to more than 150% of pre-treatment values in three patients. These abnormal blood test results may have been related to disease progression rather than treatment toxicity.

Most recently, a dose escalation study was conducted to determine the maximum tolerated dose and safety of a single dose of curcumin (65). Healthy volunteers were administered escalating doses from 500 to 12,000 mg. Seven of twenty-four subjects (30%) experienced only minimal toxicity that did not appear to be dose-related.

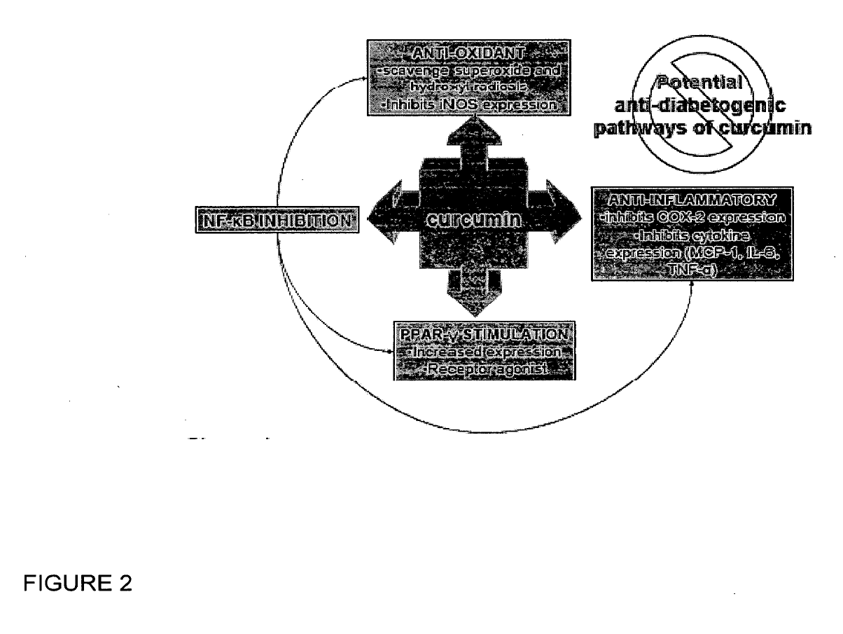

Given the fact that the pro-carcinogenic pathways that curcumin inhibits also play crucial roles with regard to insulin sensitivity and β-cell survival (FIG. 2), it would appear to have great potential for the treatment of diabetes in humans. Despite this, there is a dearth of published data regarding the effects of curcumin upon diabetes. None of the studies published examining curcumin's effect upon diabetes have been in humans; there is only a single case report which describes the effect of curcumin in a type-2 diabetic patient (66). The preclinical studies mostly utilize the diabetic streptozotocin-treated rat, a somewhat poor representative of the type-2 diabetes found in humans (67-78). Nonetheless, these studies are essentially unanimous in their findings: oral curcumin therapy prevents, delays, or ameliorates diabetes-related hyperglycemia or end-organ damage (69, 70, 74, 76, 79-82).

Celastrol is a triterpene extracted from the Chinese ‘Thunder of God Vine’ plant (Tripterygium wilfordii Hook F or TwHF). The Thunder of God Vine is well known in Chinese medicine. Celastrol is a major compound extracted from the root bark of the plant. Traditionally, the bark is crushed into a powder and incorporated into a soup, which is said to have autoimmune and anti-inflammatory properties. The chemical structure of celastrol is:

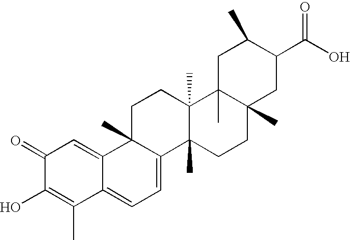

Celastrol is a potent protease inhibitor and has been reported to suppress human prostrate cancer growth in nude mice. It has been reported that celastrol potently and preferentially inhibits the chymotrypsin-like activity of a purified 20S proteasome with an IC₅₀ of 2.5 μM/L and inhibits human prostate cancer cellular 26S proteasome at 1-5 μM/L. In addition, celastrol administered to tumor-bearing nude mice at 1-3 mg/kg/d (i.p.) resulted in inhibition of tumor growth (83).

Epoxomicin, a natural product obtained from an Actinomycetes strain, is a potent and selective proteasome inhibitor (84). The synthesis of epoxomicin is well known (85) and it is commercially available (see, e.g., A.G. Scientific, San Diego, Calif.). It has been reported that epoxomicin is a potent antitumor agent and exhibits antiinflammatory activity at daily doses of between about 0.5 to about 3.0 mg/kg/d (i.p.) (84). The structure of epoxomicin is:

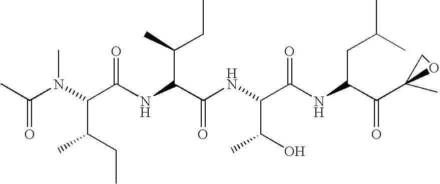

In the present invention, an “effective amount” of a selective proteasome inhibitor is an amount sufficient to effect beneficial or desired results. An effective amount can be administered to a mammal, particularly a human, in one or more doses. In terms of treatment of a mammal, an “effective amount” of a selective proteasome inhibitor is an amount sufficient to, e.g., treat, prevent, and/or ameliorate diabetes, particularly type-2 diabetes mellitus, with minor or no side effects. More particularly, an “effective amount” delivers to a subject from about 0.005 mg/kg/day to about 150 mg/kg/day of the selective proteasome inhibitor; more preferably, from about 1 mg/kg/day to about 150 mg/kg/day, such as for example from about 50 mg/kg/day to about 150 mg/kg/day. Other preferred dosages include, for example, from about 0.005 mg/kg to about 10 mg/kg; such as, from about 0.05 mg/kg to about 4 mg/kg. Thus, for example, an effective amount of the selective proteasome inhibitor is from about 0.5 mg/kg to about 2 mg/kg. In the present invention, all numerical ranges provided are intended to include at least all numbers that fall between the endpoints of the recited ranges.

Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, sex, size, and species of mammal, and like factors well known in the arts of medicine and veterinary medicine. In general, a suitable dose of one of the materials (selective proteasome inhibitor) identified in a method according to the invention will be that amount of the material, which is the lowest dose effective to produce the desired effect. The effective dose of such a material according to the invention may be administered as two, three, four, five, six or more sub-doses, administered separately at appropriate intervals throughout the day. Preferably, however, the material is administered in a once-a-day oral dosage form.

Non-limiting examples of effective once-a-day oral dosages include from about 1 g/day to about 18 g/day, such as for example from about 5 g/day to about 15 g/day, including 3 g/day, 9 g/day, and 18 g/day. Another preferred once-a-day oral dosage range is from about 1 g/day to about 1.5 g/day.

A selective proteasome inhibitor according to the present invention may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, sublingual, intramuscular, intravenous, intraarterial, intrathecal, or intralymphatic. Further, a selective proteasome inhibitor may be administered in any combination with each other and/or in conjunction with other treatments. A selective proteasome inhibitor of the invention may be encapsulated or otherwise protected against gastric or other secretions, if desired.

While it is possible for a selective proteasome inhibitor to be administered alone, it is preferable to administer the selective proteasome inhibitor as a pharmaceutical formulation (composition). The pharmaceutically acceptable compositions comprise one or more of the selective proteasome inhibitors of the present invention as an active ingredient in admixture with one or more pharmaceutically-acceptable carriers and, optionally, one or more other compounds, drugs, ingredients and/or materials. Regardless of the route of administration selected, the selective proteasome inhibitors of the present invention are formulated into pharmaceutically-acceptable dosage forms by conventional methods known to those of skill in the art. See, e.g., Remington's Pharmaceutical Sciences (Mack Publishing Co., Easton, Pa.).

In the present invention, the selective proteasome inhibitors may be co-administered with one or more so-called first line drugs for treating diabetes. As used herein, “co-administration” includes delivering two or more actives in a single unit dose, simultaneously delivering two or more actives in different unit doses (e.g., taking two tablets at the same time) or delivering two or more actives in different unit doses over a pre-determined, clinically relevant period of time. Non-limiting examples of classes of such first line drugs include α-glucosidase inhibitors, biquanides, insulins, meglitinides, sulfonylureas, thiazolidiniones, dipeptidyl peptidase (PPD-4) inhibitors, glucagon-like peptide (GLP-1) analogs, and combinations thereof, such as combinations of sulfonylurea/biquanide or thiazolidinedione/biquanide.

Non-limiting examples of α-glucosidase inhibitors include acarbose and miglitol. A non-limiting example of a biguanide is Metformin. Non-limiting examples of the meglitinides include nateglinide and repaglinide. Non-limiting examples of sulfonylureas include acetohexamide, chlorpropamide, glipizide, glipizide extended release, glyburide, tolazamide, and tolbutamide. Non-limiting examples of thiazolidinediones include pioglitazone and rosiglitazone. Non-limiting examples of PPD-4 inhibitors include sitagliptin and vildagliptin. Non-limiting examples of glucagon-like peptide (GLP-1) analogs include exenatide and livaglutide.

Pharmaceutically acceptable carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each carrier used in a composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutically acceptable compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical formulations (compositions) suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administration comprise one or more selective proteasome inhibitors in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents, which delay absorption.

Formulations for rectal administration may be presented as a suppository, which maybe prepared by mixing one or more active ingredient(s) with one or more suitable nonirritating carriers which are solid at room temperature, but liquid at body temperature and, therefore, will melt in the rectum cavity and release the active compound.

In some cases, in order to prolong the effect of a selective proteasome inhibitor, it is desirable to slow its absorption from subcutaneous or intramuscular injection. This may be accomplished by the use of a liquid suspension of crystalline or amorphous material having poor water solubility.

The rate of absorption of the selective proteasome inhibitor then depends upon its rate of dissolution, which in turn, may depend upon crystal size and crystalline form. Alternatively, delayed absorption of a parenterally-administered selective proteasome inhibitor may be accomplished by dissolving or suspending the selective proteasome inhibitor in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions that are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In the present invention, the selective proteasome inhibitors and pharmaceutical compositions and unit dosage forms containing same may be used to treat, prevent and/or ameliorate the symptoms of not only diabetes, but also of its hyperglycemic complications, including for example, nerve, vascular disease, nephropathy, retinopathy, and atherosclerosis. The selective proteasome inhibitors and pharmaceutical compositions and unit dosage forms containing same may also be used to treat, prevent and/or ameliorate the symptoms of other diseases that emanate from the hyperinsulinemic/insulin resistance syndrome, including for example, hypertension and ovarian hyperandrogenism (PCOS). The selective proteasome inhibitors and pharmaceutical compositions and unit dosage forms containing same may also be used to treat, prevent and/or ameliorate the symptoms of other diseases that may be regulated directly, or indirectly, by the proteasome, such as for example, cancer.

The following examples are provided to further illustrate the compositions and methods of the present invention. These examples are illustrative only and are not intended to limit the scope of the invention in any way.

### EXAMPLES

Described below are several preclinical studies that elucidate the extent and potential mechanisms whereby exemplary selective proteasome inhibitors of the present invention—curcumin, epoxomicin, and celastrol—could prevent diabetes-associated hyperglycemia and inflammation in three different male mouse models of obese diabetes: 1) dietary induced obese (DIO) C57BL/6J; 2) C57BL/6J ob/ob; and 3) C57BL/Ks db/db mice.

Given curcumin's excellent safety profile, we started with a high dosage, 3% by weight dietary curcumin admixture, to assess if there would be any effect at all. This translated into a daily consumption by the mice of roughly 1.0 to 1.5 g/kg/day. The wild-type C57BL/6J mice had their curcumin added to a 35% fat by weight diet to induce obesity while the ob/ob and db/db mice had their curcumin added to a low-fat 4% by weight diet (Research Diets, New Brunswick, N.J.). The curcumin utilized was a 95% curcumin extract (C3 Complex, Sabinsa Corporation, Newark, N.J.).

Male C57BU6J mice gradually develop obesity and moderate diabetes when placed on high-fat diets, a process quite analogous to that in humans. Male C57BL/6J ob/ob mice possess a deletion of the leptin gene which produces hyperphagia, decreased metabolic rate, severe obesity, and moderate diabetes which is eventually well compensated for by pancreatic β-cell hyperplasia and hyperinsulinemia. The male C57BL/Ks db/db mice possess a leptin receptor gene deletion which generates a phenotype initially very similar to that of the ob/ob mice. However, the loss of leptin effect on the C57BL/Ks background is not compensated for by β-cell hyperplasia and hyperinsulinemia. At a very young age these mice become severely hyperglycemic, hyperphagic, and polydipsic. As they mature, they start to lose weight, develop nephropathy, and ultimately die around age 40 weeks from diabetic complications.

### Administration of Proteasome Inhibitor Compounds Significantly Improves Glycemic Status and Insulin Sensitivity in Mouse Models of Obesity-Related Diabetes

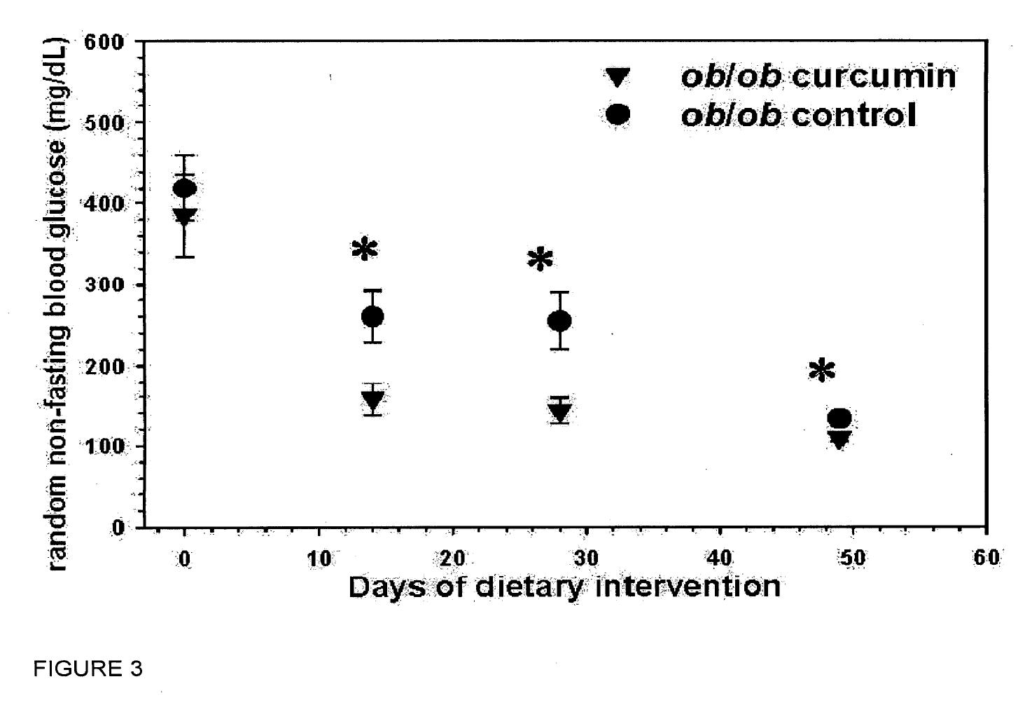

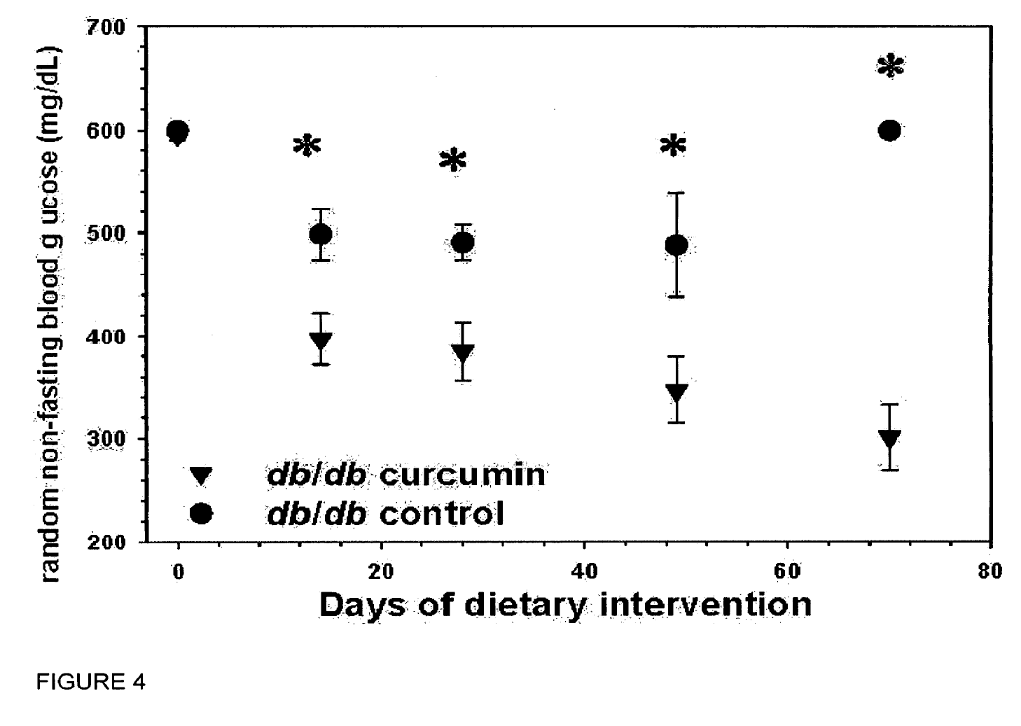

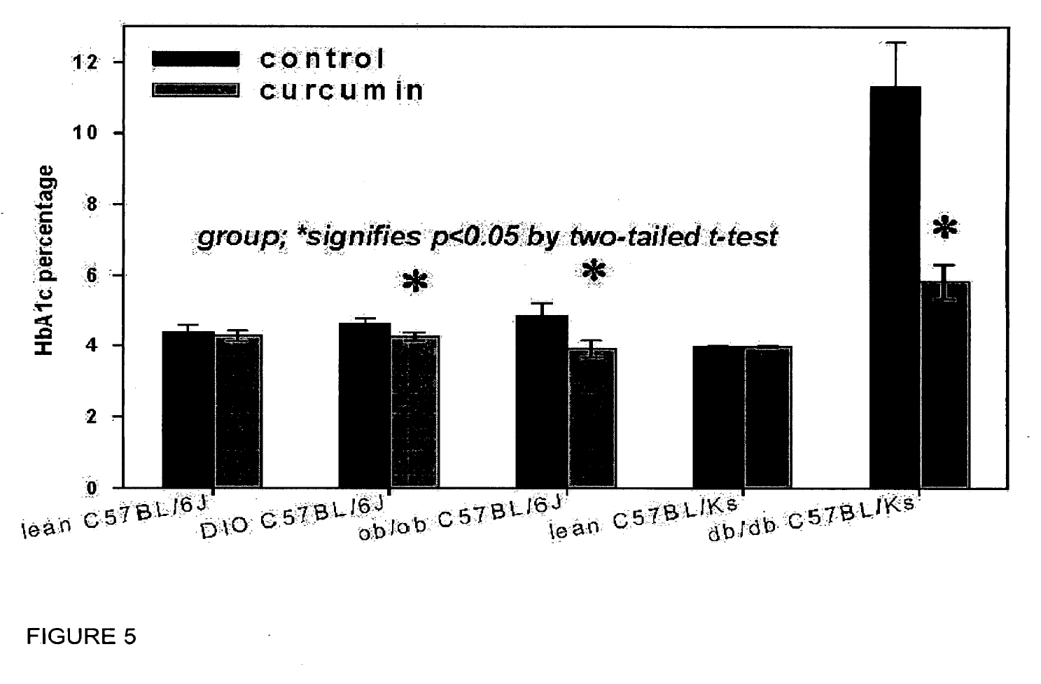

We determined that 3% dietary curcumin induces significant decreases in random fed glucose (FIGS. 3, 4) and HbA1c levels (FIG. 5) in all three diabetic mice categories as early as 2-3 weeks respectively.

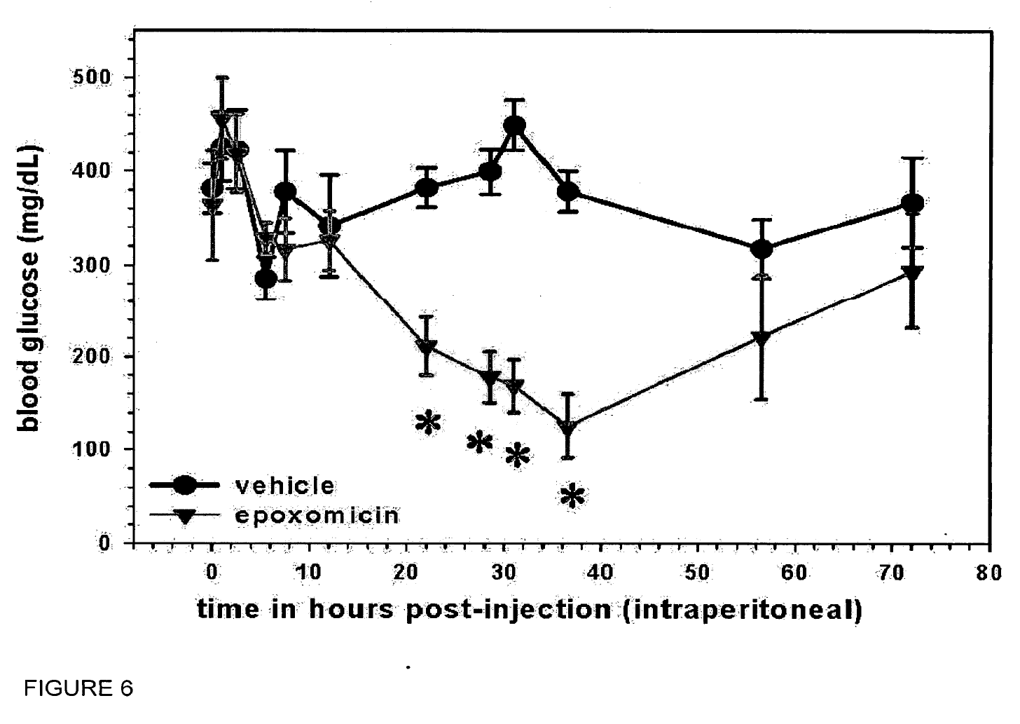

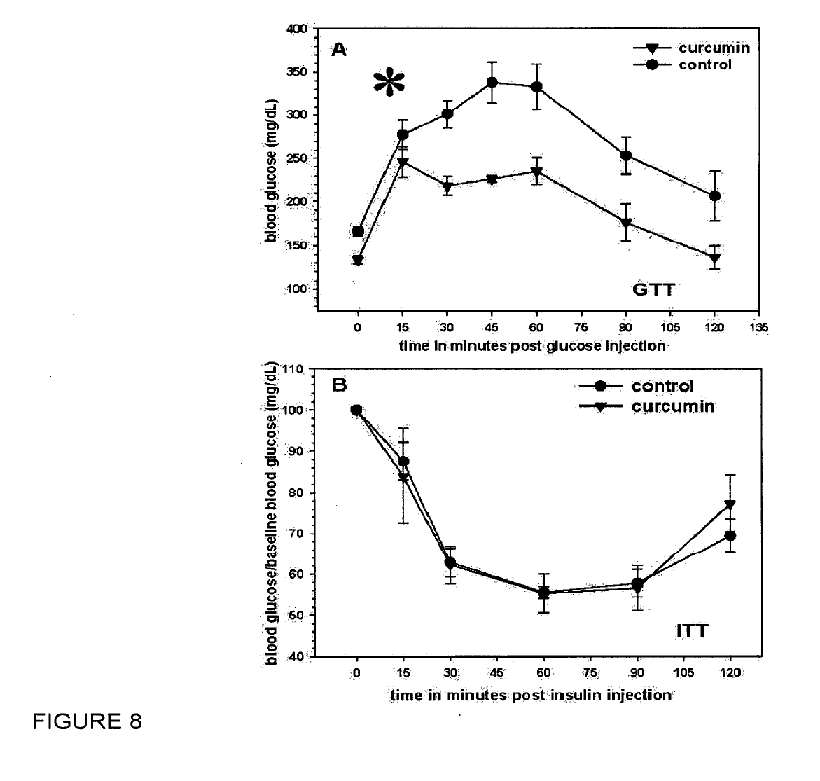

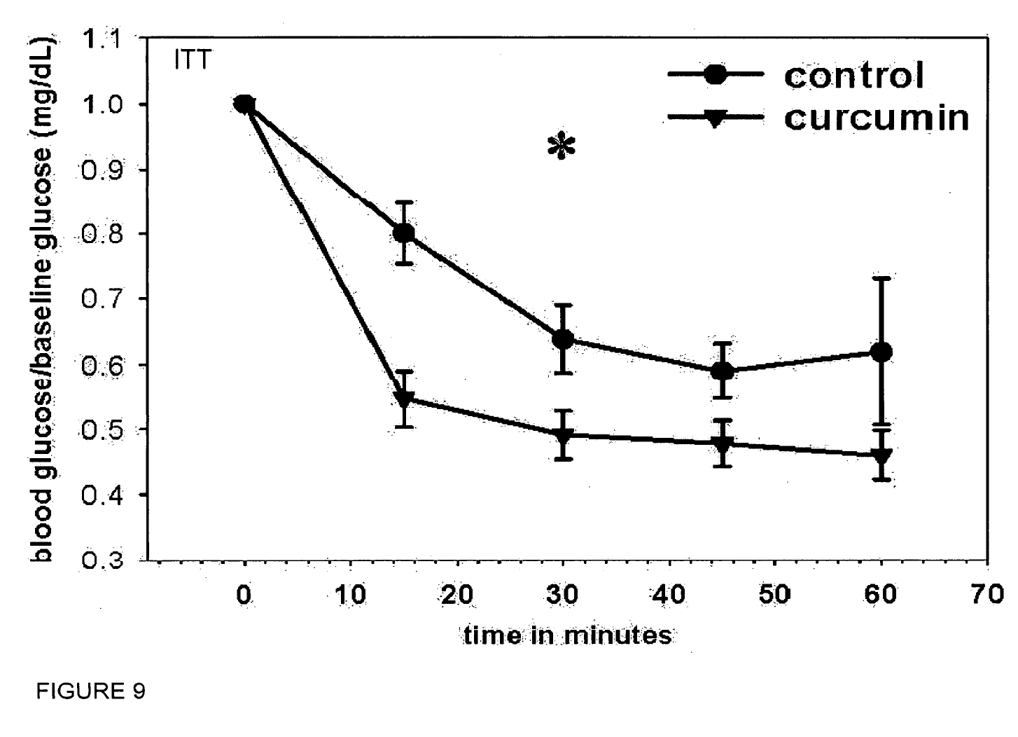

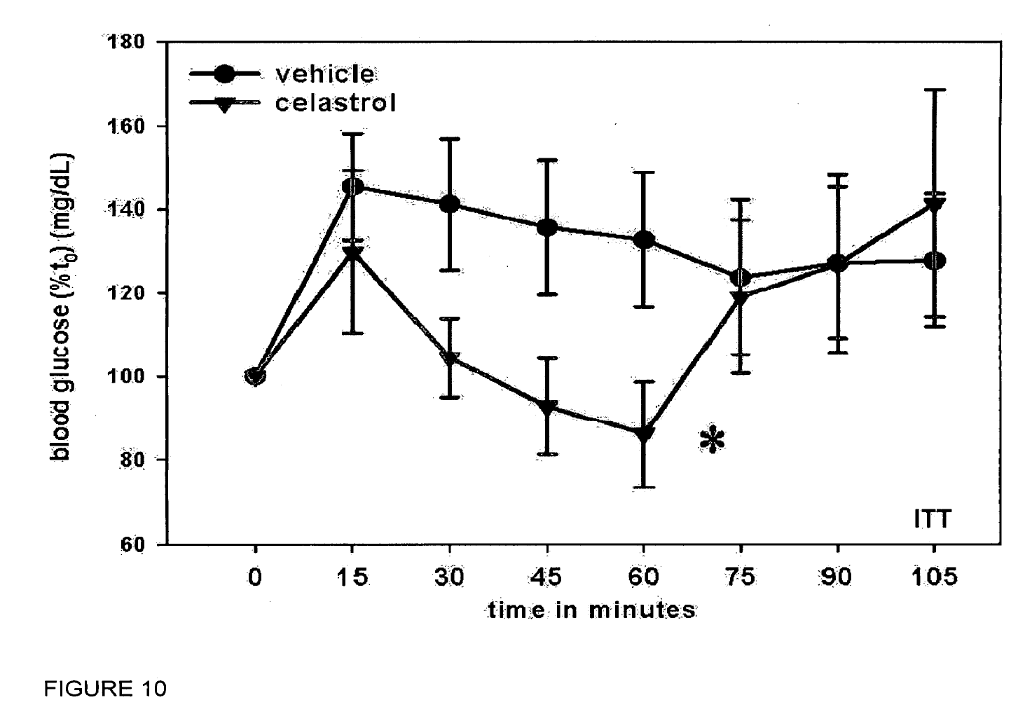

In addition, approximately 10 hours after a single intraperitoneal injection of the proteasome inhibitors epoxomicin (0.1 mg/kg) or celastrol (3 mg/kg), correction of hyperglycemia was noted in male db/db mice (FIG. 6, 7). This effect was noted to last for at least 48 hours post-injection. Mice receiving vehicle in the celastrol and epoxomicin experiments were food entrained to the treatment group to avoid any acute effects on glucose levels from differences in food intake. Dietary curcumin improved glucose tolerance (FIG. 8A) but not insulin tolerance (FIG. 8B) in male DIO mice. Insulin tolerance however, was demonstrated in the male ob/ob mice by a decreased area under the curve (AUC) during an insulin tolerance test (ITT) (FIG. 9). Twenty-four hours after a single intraperitoneal celastrol injection, improved insulin tolerance in the male db/db mice was also demonstrated by a decreased area under the ITT curve (FIG. 10).

### Curcumin has a Beneficial Effect on Body Composition

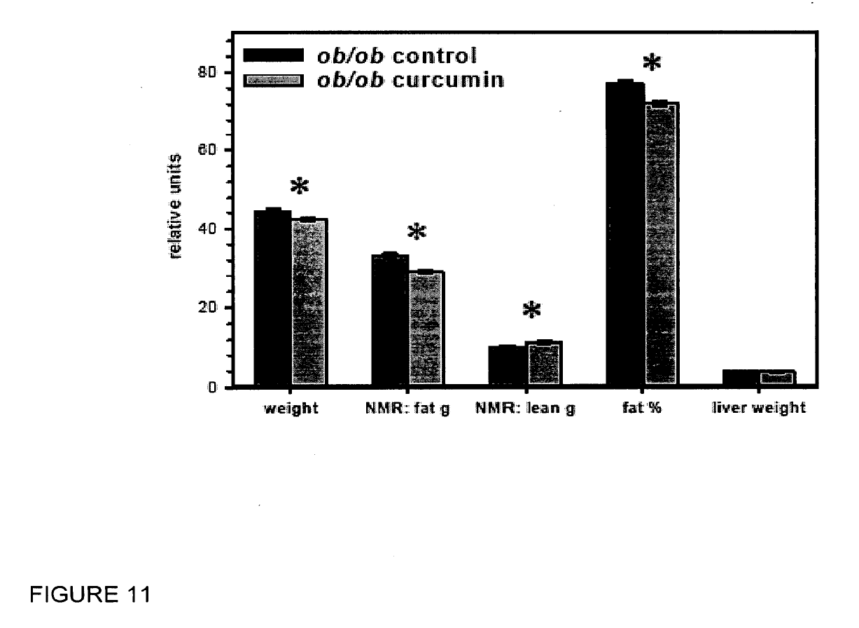

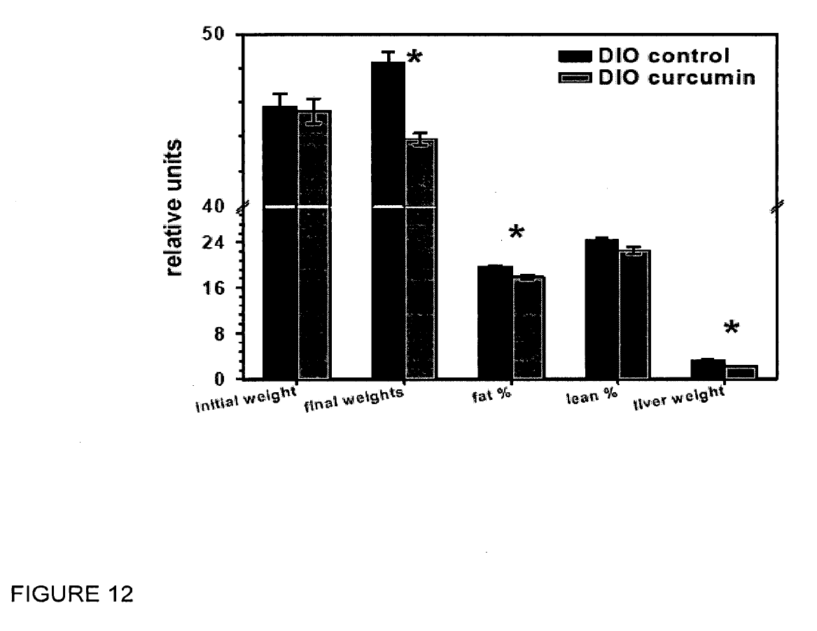

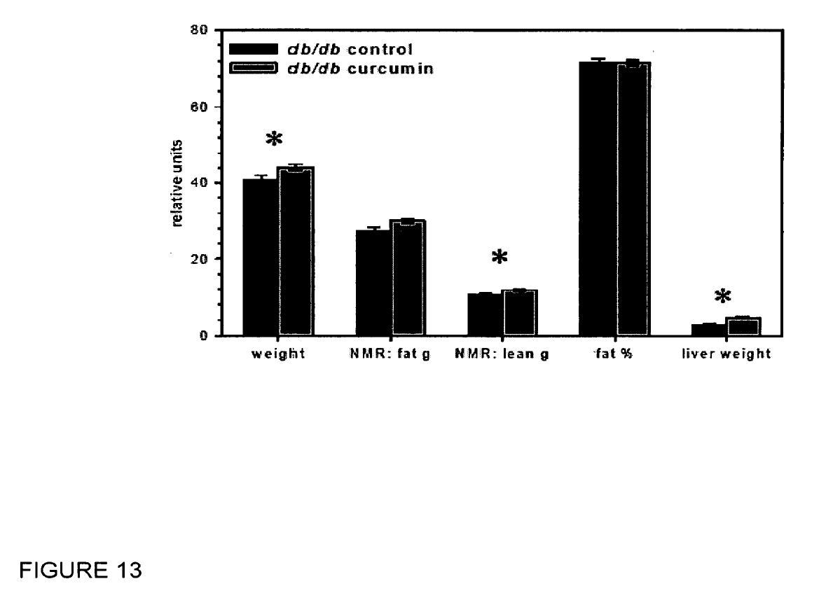

Male DIO and ob/ob mice whose food contained 3% curcumin consumed significantly more food per day than control mice, even after compensating for the percent of their food that was curcumin (not shown). Despite the increased caloric intake, the curcumin treated DIO and ob/ob mice weighed slightly but significantly less than their control cohort (FIGS. 11, 12). The C57BL/Ks db/db mice, on the other hand, actually ate less and weighed more than their control cohort, a finding consistent with the fact that they were much less diabetic and were better able to incorporate the calories they consumed (FIG. 13). Intriguingly, curcumin treatment was associated with significantly more lean mass (as determined by Bruker NMR analysis) in both male ob/ob and db/db mice (FIGS. 11, 13). The DIO and ob/ob mice manifested significantly less body fat also (FIGS. 11, 12). This may potentially stem from curcumin's ability to inhibit NF-κB, an effect which has been shown to prevent muscle loss.

### Proteasome Inhibitors Significantly Decrease Hepatic Inflammation

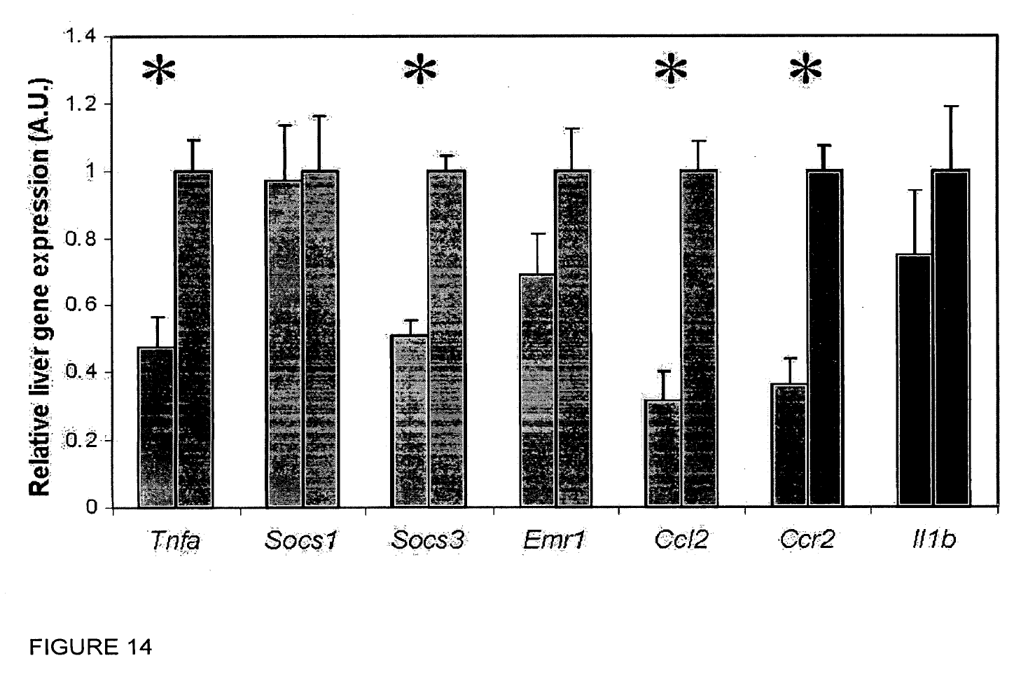

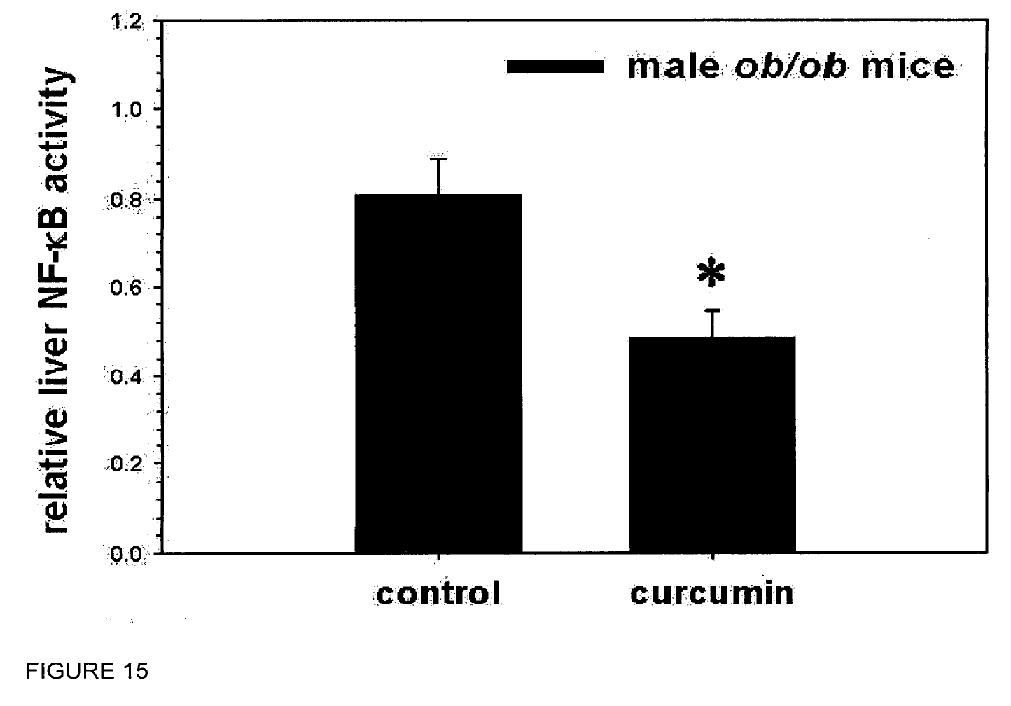

Quantitative real-time PCR (SYBR® GreenER™ qPCR Reagent System, Invitrogen, Carlsbad Calif.) on an MJ Opticon2 cycler revealed that the expression of several genes implicated in inflammatory pathways were significantly downregulated in hepatic tissue after 10 weeks of dietary curcumin in male ob/ob mice (FIG. 14). These included TNF-α, Socs-3, Ccl2 (MCP-1 gene) and Ccr2 (MCP-1 receptor gene). In addition, using a specific assay for p65 activity (TransAM™ NFkB p65 Kit, Active Motif, Carlsbad, Calif.), we noted that there was significantly less NFkB activity in liver nuclear extract samples derived from the curcumin treated ob/ob mice as compared to those derived from untreated controls (FIG. 15). Not surprisingly, the liver weights and degree of liver steatosis were significantly lower in DIO and ob/ob mice fed curcumin as compared to controls (data not shown).

### Proteasome Inhibitors Significantly Decrease Adipose Inflammation

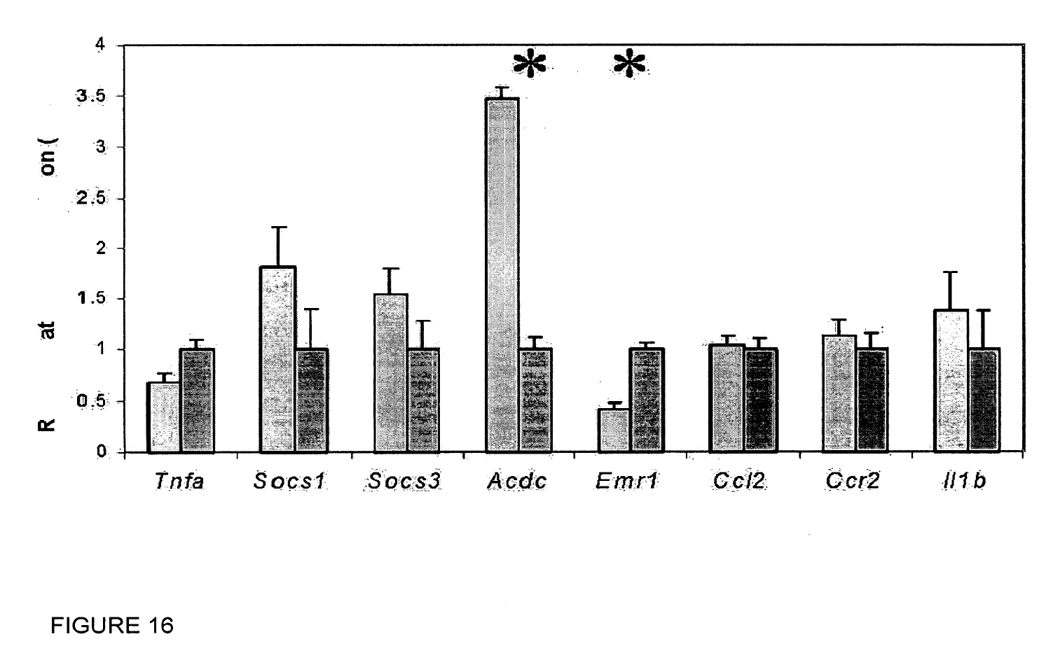

Given that the adipose tissue of obese subjects is chronically inflamed and secretes diabetogenic adipokines, we investigated the possibility that proteasome inhibition improves diabetes by decreasing adipose inflammation in obese diabetic mice. We analyzed the effect of curcumin treatment upon the expression of several genes capable of modulating the inflammatory process using quantitative real time PCR. We determined that curcumin treatment dramatically increased adipose adiponectin gene (Acdc) expression (FIG. 16). (Serum adiponectin levels were also significantly higher in the curcumin-treated ob/ob mice (not shown), corroborating the expression data and consistent with their improved findings on the ITT).

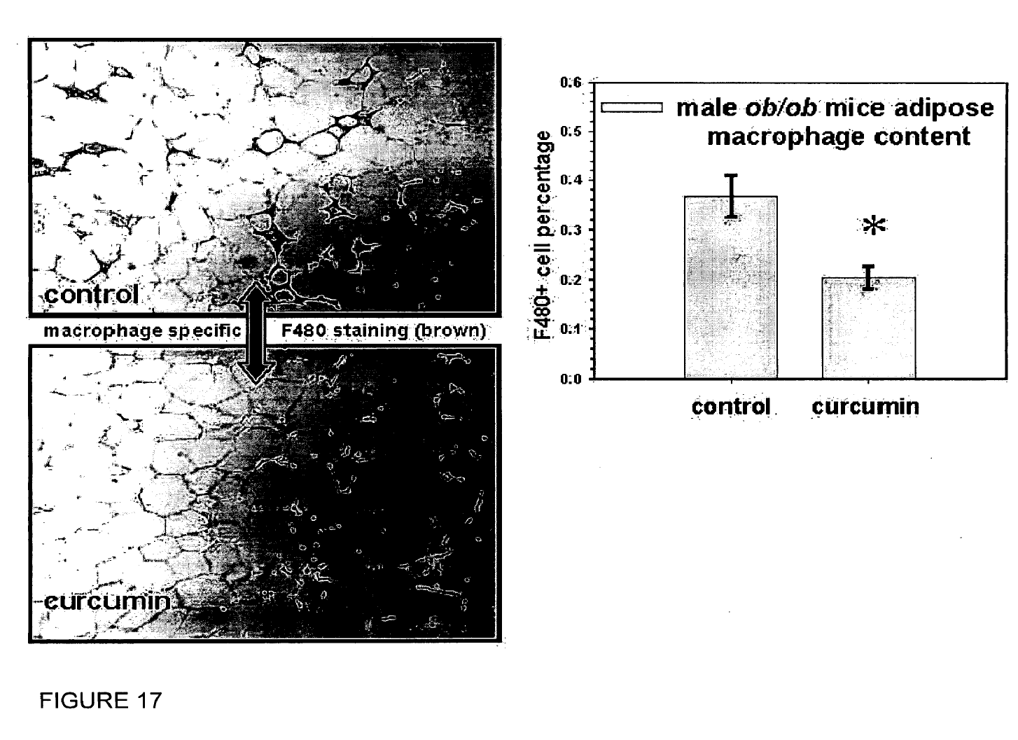

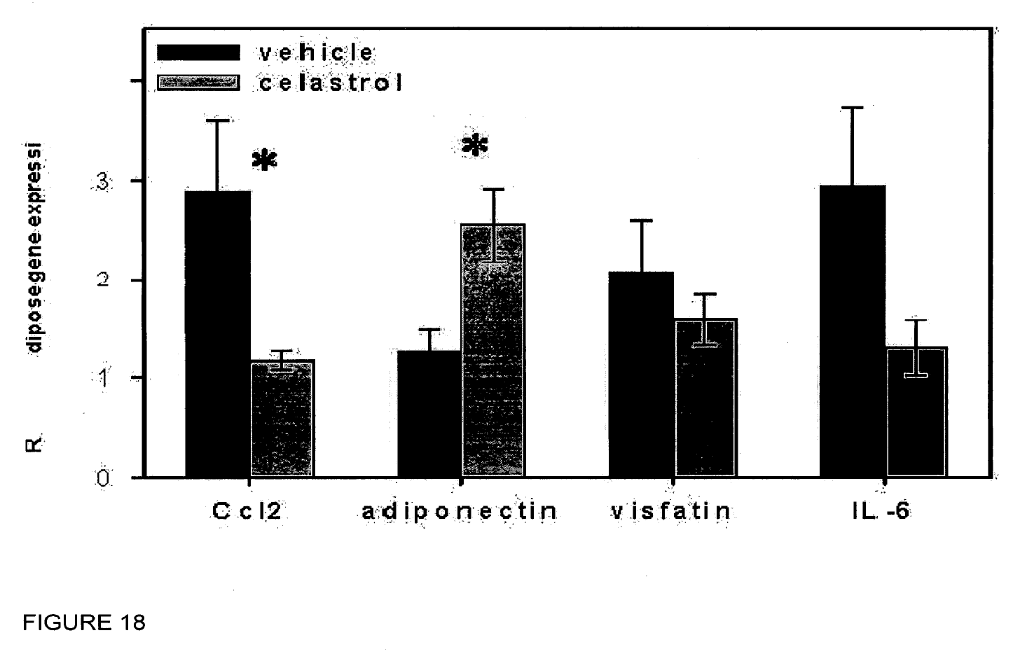

Immunohistochemistry revealed that curcumin induced a dramatic reduction in the number of macrophages present in the adipose tissue of ob/ob mice as determined by staining with a macrophage specific F480 antibody (FIG. 17). This also gibed with expression data that revealed significantly decreased macrophage-specific Emr1 (F480) expression (FIG. 16) in adipose from curcumin-fed ob/ob mice. In addition, three days of a single daily celastrol IP injection resulted in significantly decreased adipose expression of Ccl-2 and significantly increased expression of adiponectin in male db/db mice (FIG. 18).

### Proteasome Inhibitors Increase Pancreatic β-Cell Hyperplasia and Insulin Release

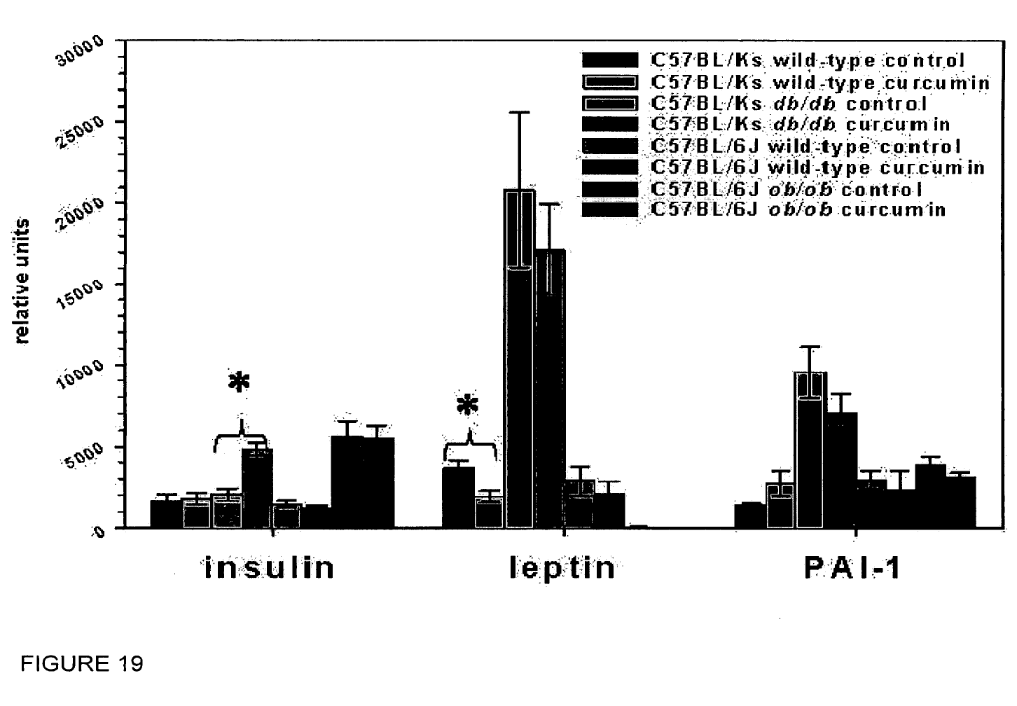

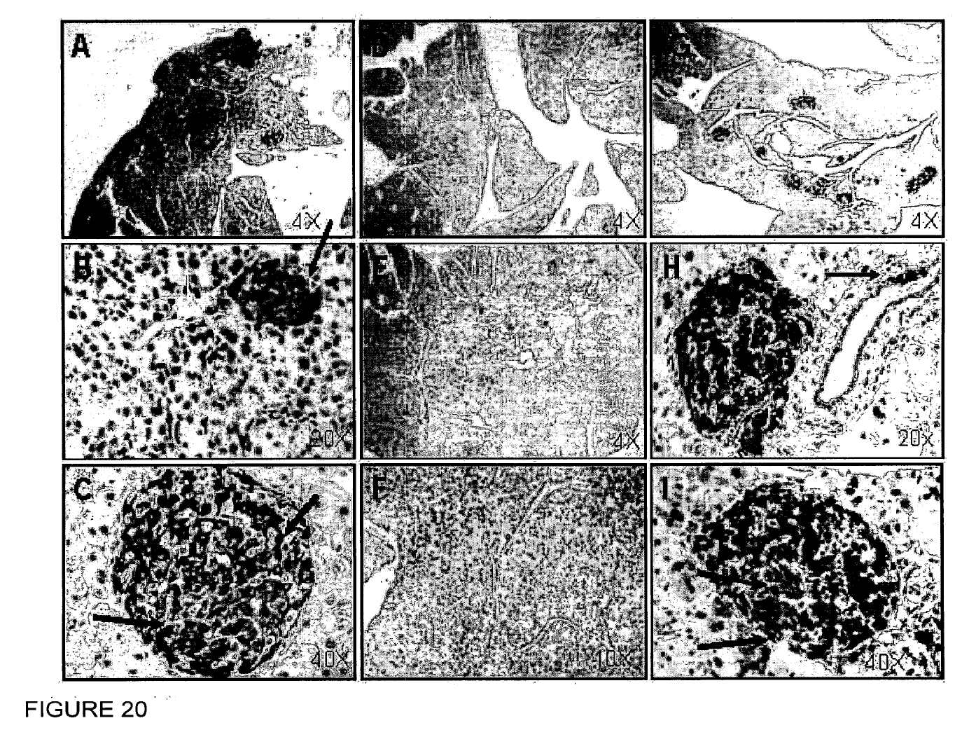

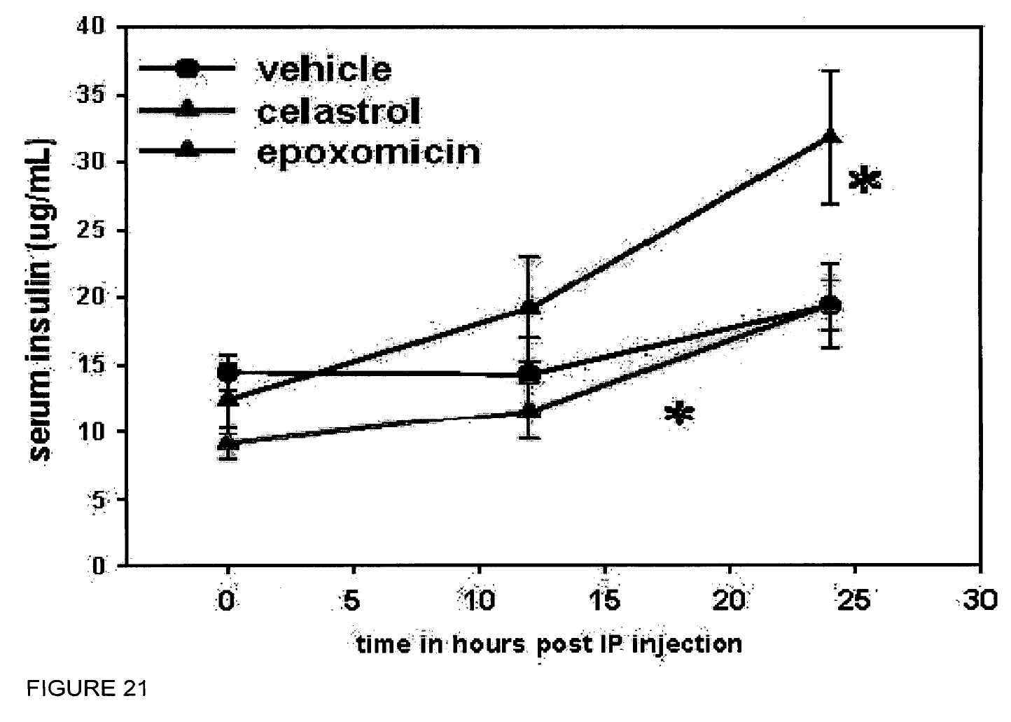

As FIGS. 19 and 20 A-C reveal, untreated ob/ob mice develop pancreatic β-cell hyperplasia and hyperinsulinemia, a phenomenon that ultimately allows them to recoup normoglycemia. The pancreatic islets in untreated C57BL/Ks db/db mice however, degenerate (FIG. 20 D-F). When C57BL/Ks db/db mice are treated with curcumin, however, their islets actually become hyperplastic (FIG. 20 G-I) and contain some proliferating β-cells as evidenced by the presence of nuclear Ks67 immunoreactivity (see arrows in FIG. 20 G-I). Not surprisingly, curcumin-treated db/db mice also exhibit hyperinsulinemia (FIG. 19) just like untreated ob/ob mice. When we IP injected C57BL/Ks db/db mice with celastrol (3 mg/kg) or epoxomicin (0.1 mg/kg), we noted significant increases in serum insulin at 24 hours post injection (FIG. 21), a time point corresponding to when the peak hypoglycemic effects induced by these injections occurred.

### Proteasome Inhibitors Alter β-Cell PTEN, Foxo3a, and INGAP Expression

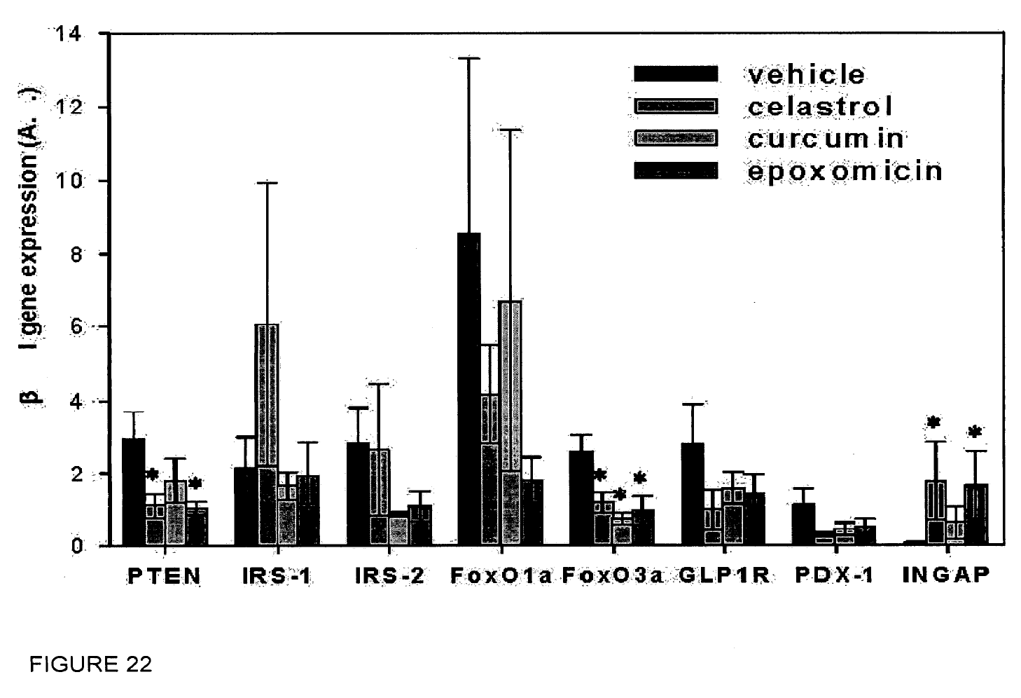

When we selectively isolated the β-cells from male db/db pancreata by collagenase digestion and centrifugation, we noted that β-cells derived from mice treated with proteasome inhibitors had significant decreases in the expression of PTEN and Foxo3a, but increased expression of INGAP (Islet Neogenesis Associated Protein) (FIG. 22). The directionality of these three transcription factor modulations is consistent with beneficial effects on diabetes and β-cell proliferation (86, 87).

### Proteasome Inhibitors Increase Proliferation of the β-Cell Line INS-1

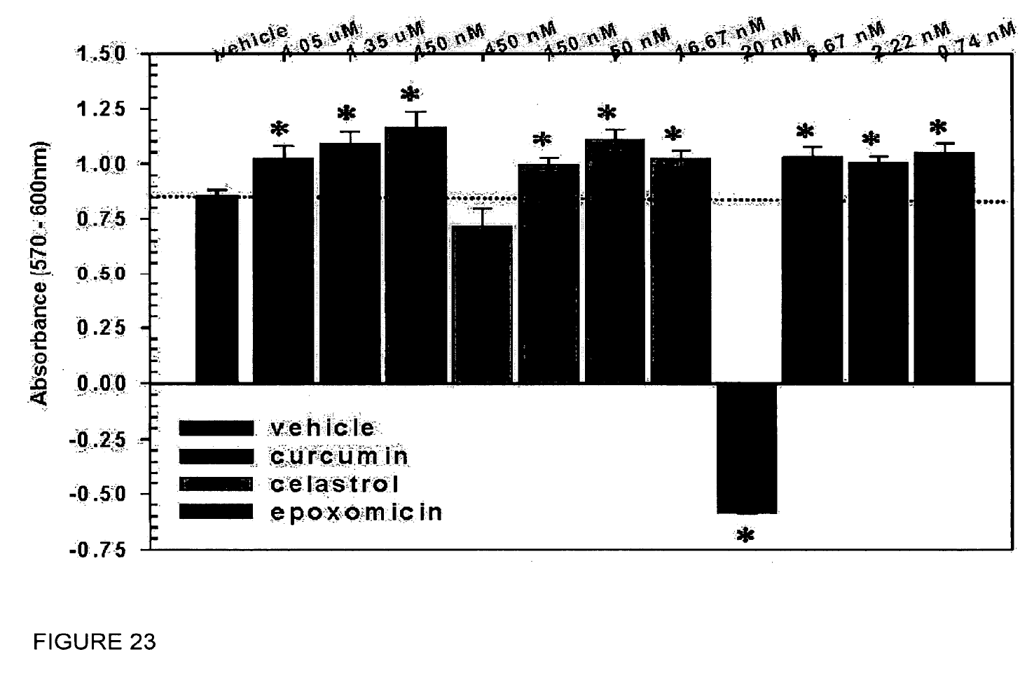

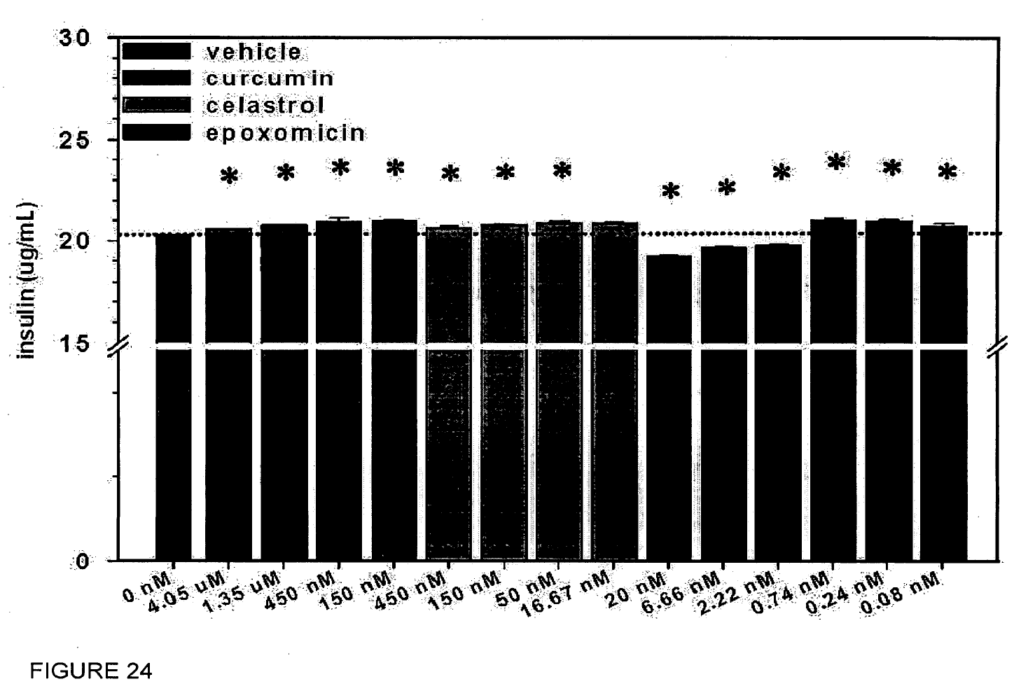

To follow-up on the potential ability of proteasome inhibition to improve β-cell function, we performed experiments using the rat β-cell line Ins-1. We determined that the number of viable Ins-1 cells (CellTiter-Blue Cell Viabillity Assay, Promega, Madison, Wis.) after 24 hours in culture with varying concentrations of proteasome inhibitors was increased, except at the highest concentrations of celastrol and epoxomicin, which proved cytotoxic (FIG. 23). When Ins-1 cells were cultured overnight in serum-free RPMI media containing varying concentrations of proteasome inhibitors, insulin secretion was significantly increased by proteasome inhibition, except at the highest concentrations of epoxomicin, which again proved cytotoxic (FIG. 24).

In summary, our studies reveal that administration of selective proteasome inhibitors to three different mouse models of diabetic obesity significantly diminishes their tissue inflammation and greatly improves their hyperglycemia. Given that selective proteasome inhibition has the ability to affect every cell in the body directly, it is not surprising that the mechanisms by which selective proteasome inhibition improves diabetes appear to be multiple; although the most impressive effect is that upon the pancreatic β-cell. It is worth emphasizing the fact that, although bortezomib is marketed as an anti-cancer, pro-apoptotic drug, the selective proteasome inhibitors in this study actually fostered β-cell proliferation until a cytotoxic concentration was reached. Therefore, it is possible that proteasome inhibitors may improve diabetes in humans at doses less than that needed for cancer treatment.

### CITED DOCUMENTS

The following documents, cited above, are incorporated by reference as if recited in full herein:
1. Taha D, Umpaichitra V, Banerji M A, Castells S 2006 Type 2 diabetes mellitus in African-American adolescents: impaired beta-cell function in the face of severe insulin resistance. J Pediatr Endocrinol Metab 19:135-142.
2. Vivian E M 2006 Type 2 diabetes in children and adolescents—the next epidemic? Curr Med Res Opin 22:297-306.
3. Ahren B 2005 Type 2 diabetes, insulin secretion and beta-cell mass. Curr Mol Med 5:275-286.
4. Golay A, Ybarra J 2005 Link between obesity and type 2 diabetes. Best Pract Res Clin Endocrinol Metab 19:649-663.
5. Matsuzawa Y 2006 The metabolic syndrome and adipocytokines. FEBS Lett.
6. Permutt M A, Chiu K, Ferrer J, Glaser B, Inoue H, Nestorowicz A, Stanley C A, Tanizawa Y 1998 Genetics of type II diabetes. Recent Prog Horm Res 53:201-216.
7. Friday R P, Trucco M, Pietropaolo M 1999 Genetics of Type 1 diabetes mellitus. Diabetes Nutr Metab 12:3-26.
8. Zhang P, Engelgau M M, Valdez R, Benjamin S M, Cadwell B, Narayan K M 2003 Costs of screening for pre-diabetes among US adults: a comparison of different screening strategies. Diabetes Care 26:2536-2542.
9. Roglic G, Unwin N, Bennett P H, Mathers C, Tuomilehto J, Nag S, Connolly V, King H 2005 The burden of mortality attributable to diabetes: realistic estimates for the year 2000. Diabetes Care 28:2130-2135.
10. Engelgau M M, Geiss L S, Saaddine J B, Boyle J P, Benjamin S M, Gregg E W, Tierney E F, Rios— Burrows N, Mokdad A H, Ford E S, Imperatore G, Narayan K M 2004 The evolving diabetes burden in the United States. Ann Intern Med 140:945-950.
11. Baumeister W, Walz J, Zuhl F, Seemuller E 1998 The proteasome: paradigm of a self-compartmentalizing protease. Cell 92:367-380.
12. Lowe J, Stock D, Jap B, Zwickl P, Baumeister W, Huber R 1995 Crystal structure of the 20S proteasome from the archaeon T. acidophilum at 3.4 A resolution. Science 268:533-539.
13. Groll M, Ditzel L, Lowe J, Stock D, Bochtler M, Bartunik H D, Huber R 1997 Structure of 20S proteasome from yeast at 2.4 A resolution. Nature 386:463-471.
14. Walz J, Erdmann A, Kania M, Typke D, Koster A J, Baumeister W 1998 26S proteasome structure revealed by three-dimensional electron microscopy. J Struct Biol 121:19-29.
15. Hideshima T, Chauhan D, Richardson P, Mitsiades C, Mitsiades N, Hayashi T, Munshi N, Dang L, Castro A, Palombella V, Adams J, Anderson K C 2002 NF-kappa B as a therapeutic target in multiple myeloma. J Biol Chem 277:16639-16647.
16. Shen J, Reis J, Morrison D C, Papasian C, Raghavakaimal S, Kolbert C, Qureshi A A, Vogel S N, Qureshi N 2006 Key inflammatory signaling pathways are regulated by the proteasome. Shock 25:472-484.
17. Bai J, Sui J, Demirjian A, Vollmer C M, Jr., Marasco W, Callery M P 2005 Predominant Bcl-XL knockdown disables antiapoptotic mechanisms: tumor necrosis factor-related apoptosis-inducing ligand-based triple chemotherapy overcomes chemoresistance in pancreatic cancer cells in vitro. Cancer Res 65:2344-2352.
18. Mortenson M M, Schlieman M G, Virudachalam S, Lara P N, Gandara D G, Davies A M, Bold R J 2005 Reduction in BCL-2 levels by 26S proteasome inhibition with bortezomib is associated with induction of apoptosis in small cell lung cancer. Lung Cancer 49:163-170.
19. Henninger N, Sicard K M, Bouley J, Fisher M, Stagliano N E 2006 The proteasome inhibitor VELCADE reduces infarction in rat models of focal cerebral ischemia. Neurosci Lett 398:300-305.
20. Marfella R, D'Amico M, Esposito K, Baldi A, Di Filippo C, Siniscalchi M, Sasso F C, Portoghese M, Cirillo F, Cacciapuoti F, Carbonara O, Crescenzi B, Baldi F, Ceriello A, Nicoletti G F, D'Andrea F, Verza M, Coppola L, Rossi F, Giugliano D 2006 The ubiquitin-proteasome system and inflammatory activity in diabetic atherosclerotic plaques: effects of rosiglitazone treatment. Diabetes 55:622-632.
21. Ostrowska H, Kruszewski K, Kasacka I 2006 Immuno-proteasome subunit LMP7 is up-regulated in the ischemic kidney in an experimental model of renovascular hypertension. Int J Biochem Cell Biol 38:1778-1785.
22. Anan A, Baskin-Bey E S, Isomoto H, Mott J L, Bronk S F, Albrecht J H, Gores G J 2006 Proteasome inhibition attenuates hepatic injury in the bile duct-ligated mouse. Am J Physiol Gastrointest Liver Physiol 291:G709-716.
23. Cinti S, Mitchell G, Barbatelli G, Murano I, Ceresi E, Faloia E, Wang S, Fortier M, Greenberg A S, Obin M S 2005 Adipocyte death defines macrophage localization and function in adipose tissue of obese mice and humans. J Lipid Res 46:2347-2355.
24. Kanda H, Tateya S, Tamori Y, Kotani K, Hiasa K, Kitazawa R, Kitazawa S, Miyachi H, Maeda S, Egashira K, Kasuga M 2006 MCP-1 contributes to macrophage infiltration into adipose tissue, insulin resistance, and hepatic steatosis in obesity. J Clin Invest 116:1494-1505.
25. Ferroni P, Guagnano M T, Manigrasso M R, Ciabattoni G, Davi G 2005 Increased plasminogen activator inhibitor-1 levels in android obesity: correlation with oxidative stress. J Thromb Haemost 3:1086-1087.
26. Deans K A, Sattar N 2006 “Anti-inflammatory” drugs and their effects on type 2 diabetes. Diabetes Technol Ther 8:18-27.
27. Sjoholm A, Nystrom T 2006 Inflammation and the etiology of type 2 diabetes. Diabetes Metab Res Rev 22:4-10.
28. Wu L, Nicholson W, Knobel S M, Steffner R J, May J M, Piston D W, Powers A C 2004 Oxidative stress is a mediator of glucose toxicity in insulin-secreting pancreatic islet cell lines. J Biol Chem 279:12126-12134.
29. Bastard J P, Maachi M, Lagathu C, Kim M J, Caron M, Vidal H, Capeau J, Feve B 2006 Recent advances in the relationship between obesity, inflammation, and insulin resistance. Eur Cytokine Netw 17:4-12.
30. Chen H 2006 Cellular inflammatory responses: novel insights for obesity and insulin resistance. Pharmacol Res 53:469-477.
31. Feve B, Bastard J P, Vidal H 2006 (Relationship between obesity, inflammation and insulin resistance: new concepts). C R Biol 329:587-597; discussion 653-585.
32. de Rekeneire N, Peila R, Ding J, Colbert L H, Visser M, Shorr R I, Kritchevsky S B, Kuller L H, Strotmeyer E S, Schwartz A V, Vellas B, Harris T B 2006 Diabetes, hyperglycemia, and inflammation in older individuals: the health, aging and body composition study. Diabetes Care 29:1902-1908.
33. Esposito K, Giugliano G, Scuderi N, Giugliano D 2006 Role of adipokines in the obesity-inflammation relationship: the effect of fat removal. Plast Reconstr Surg 118:1048-1057; discussion 1058-1049.
34. Hundal R S, Petersen K F, Mayerson A B, Randhawa P S, Inzucchi S, Shoelson S E, Shulman G I 2002 Mechanism by which high-dose aspirin improves glucose metabolism in type 2 diabetes. J Clin Invest 109:1321-1326.
35. Whitehead J P, Richards A A, Hickman I J, Macdonald G A, Prins J B 2006 Adiponectin—a key adipokine in the metabolic syndrome. Diabetes Obes Metab 8:264-280.
36. Buckingham R E 2005 Thiazolidinediones: Pleiotropic drugs with potent anti-inflammatory properties for tissue protection. Hepatol Res.
37. Takebayashi K, Matsumoto S, Wakabayashi S, Inukai Y, Matsutomo R, Aso Y, Inukai T 2005 The effect of low-dose atorvastatin on circulating monocyte chemoattractant protein-1 in patients with type 2 diabetes complicated by hyperlipidemia. Metabolism 54:1225-1229.
38. Norlin S, Ahlgren U, Edlund H 2005 Nuclear factor-{kappa}B activity in {beta}-cells is required for glucose-stimulated insulin secretion. Diabetes 54:125-132.
39. Balasubramanyam M, Sampathkumar R, Mohan V 2005 Is insulin signaling molecules misguided in diabetes for ubiquitin-proteasome mediated degradation? Mol Cell Biochem 275:117-125.
40. Briaud I, Dickson L M, Lingohr M K, McCuaig J F, Lawrence J C, Rhodes C J 2005 Insulin receptor substrate-2 proteasomal degradation mediated by a mammalian target of rapamycin (mTOR)-induced negative feedback down-regulates protein kinase B-mediated signaling pathway in beta-cells. J Biol Chem 280:2282-2293.
41. Di Paolo S, Teutonico A, Leogrande D, Capobianco C, Schena P F 2006 Chronic inhibition of mammalian target of rapamycin signaling downregulates insulin receptor substrates 1 and 2 and AKT activation: A crossroad between cancer and diabetes? J Am Soc Nephrol 17:2236-2244.
42. Kitamura Y I, Kitamura T, Kruse J P, Raum J C, Stein R, Gu W, Accili D 2005 FoxO1 protects against pancreatic beta cell failure through NeuroD and MafA induction. Cell Metab 2:153-163.
43. Harmon J S, Stein R, Robertson R P 2005 Oxidative stress-mediated, post-translational loss of MafA protein as a contributing mechanism to loss of insulin gene expression in glucotoxic beta cells. J Biol Chem 280:11107-11113.
44. Christie M J 1995 Molecular and functional diversity of K⁺ channels. Clin Exp Pharmacol Physiol 22:944-951.
45. Inoue H, Ferrer J, Warren-Perry M, Zhang Y, MilIns H, Turner R C, Elbein S C, Hampe C L, Suarez B K, Inagaki N, Seino S, Permutt M A 1997 Sequence variants in the pancreatic islet beta-cell inwardly rectifying K+ channel Kir6.2 (Bir) gene: identification and lack of role in Caucasian patients with NIDDM. Diabetes 46:502-507.
46. Huopio H, Otonkoski T, Vauhkonen I, Reimann F, Ashcroft F M, Laakso M 2003 A new subtype of autosomal dominant diabetes attributable to a mutation in the gene for sulfonylurea receptor 1. Lancet 361:301-307.
47. Sperling M A, Menon R K 2004 Differential diagnosis and management of neonatal hypoglycemia. Pediatr Clin North Am 51:703-723.
48. Gloyn A L, Siddiqui J, Ellard S 2006 Mutations in the genes encoding the pancreatic beta-cell KATP channel subunits Kir6.2 (KCNJ11) and SUR1 (ABCC8) in diabetes mellitus and hyperinsulinism. Hum Mutat 27:220-231.
49. Tammaro P, Proks P, Ashcroft F M 2006 Functional effects of naturally occurring KCNJ11 mutations causing neonatal diabetes on cloned cardiac KATP channels. J Physiol 571:3-14.
50. Lin Y W, MacMullen C, Ganguly A, Stanley C A, Shyng S L 2006 A novel KCNJ11 mutation associated with congenital hyperinsulinism reduces the intrinsic open probability of beta-cell ATP-sensitive potassium channels. J Biol Chem 281:3006-3012.
51. Slingerland A S, Hattersley A T 2005 Mutations in the Kir6.2 subunit of the KATP channel and permanent neonatal diabetes: new insights and new treatment. Ann Med 37:186-195.
52. Yan F F, Lin C W, Cartier E A, Shyng S L 2005 Role of ubiquitin-proteasome degradation pathway in biogenesis efficiency of {beta}-cell ATP-sensitive potassium channels. Am J Physiol Cell Physiol 289:C1351-1359.
53. Araujo C C, Leon L L 2001 Biological activities of Curcuma longa L. Mem Inst Oswaldo Cruz 96:723-728.
54. Huang M T, Ma W, Lu Y P, Chang R L, Fisher C, Manchand P S, Newmark H L, Conney A H 1995 Effects of curcumin, demethoxycurcumin, bisdemethoxycurcumin and tetrahydrocurcumin on 12-Otetradecanoylphorbol-13-acetate-induced tumor promotion. Carcinogenesis 16:2493-2497.
55. Sreejayan, Rao M N 1994 Curcuminoids as potent inhibitors of lipid peroxidation. J Pharm Pharmacol 46:1013-1016.
56. Sharma R A, Euden S A, Platton S L, Cooke D N, Shafayat A, Hewitt H R, Marczylo T H, Morgan B, Hemingway D, Plummer S M, Pirmohamed M, Gescher A J, Steward W P 2004 Phase I clinical trial of oral curcumin: biomarkers of systemic activity and compliance. Clin Cancer Res 10:6847-6854.
57. Cheng A L, Hsu C H, Lin J K, Hsu M M, Ho Y F, Shen T S, Ko J Y, Lin J T, Lin B R, Ming-Shiang W, Yu H S, Jee S H, Chen G S, Chen T M, Chen C A, Lai M K, Pu Y S, Pan M H, Wang Y J, Tsai C C, Hsieh C Y 2001 Phase I clinical trial of curcumin, a chemopreventive agent, in patients with high-risk or pre-malignant lesions. Anticancer Res 21:2895-2900.
58. Deshpande S S, Ingle A D, Maru G B 1998 Chemopreventive efficacy of curcumin-free aqueous turmeric extract in 7,12-dimethylbenz(a)anthracene-induced rat mammary tumorigenesis. Cancer Lett 123:35-40.
59. Chan M M, Huang H I, Fenton M R, Fong D 1998 In vivo inhibition of nitric oxide synthase gene expression by curcumin, a cancer preventive natural product with anti inflammatory properties. Biochem Pharmacol 55:1955-1962
60. Pereira M A, Grubbs C J, Barnes L H, Li H, Olson G R, Eto I, Juliana M, Whitaker L M, Kelloff G J, Steele V E, Lubet R A 1996 Effects of the phytochemicals, curcumin and quercetin, upon azoxymethane-induced colon cancer and 7,12-dimethylbenz(a)anthracene-induced mammary cancer in rats. Carcinogenesis 17:1305-1311.
61. Wahlstrom B, Blennow G 1978 A study on the fate of curcumin in the rat. Acta Pharmacol Toxicol (Copenh) 43:86-92.
62. 1996 Clinical development plan: curcumin. J Cell Biochem Suppl 26:72-85.
63. 1993 NTP Toxicology and Carcinogenesis Studies of Turmeric Oleoresin (CAS No. 8024-37-1) (Major Component 79%-85% Curcumin, CAS No. 458-37-7) in F344/N Rats and B6C3F1 Mice (Feed Studies). Natl Toxicol Program Tech Rep Ser 427:1-275.
64. Deodhar S D, Sethi R, Srimal R C 1980 Preliminary study on antirheumatic activity of curcumin (diferuloyl methane). Indian J Med Res 71:632-634.
65. Lao C D, Ruffin M Tt, Normolle D, Heath D D, Murray S I, Bailey J M, Boggs M E, Crowell J, Rock C L, Brenner D E 2006 Dose Escalation of a Curcuminoid Formulation. BMC Complement Altern Med 6:10.
66. Srinivasan M 1972 Effect of curcumin on blood sugar as seen in a diabetic subject. Indian J Med Sci 26:269-270.
67. Babu P S, Srinivasan K 1995 Influence of dietary curcumin and cholesterol on the progression of experimentally induced diabetes in albino rat. Mol Cell Biochem 152:13-21.
68. Babu P S, Srinivasan K 1997 Hypolipidemic action of curcumin, the active principle of turmeric (Curcuma longa) in streptozotocin induced diabetic rats. Mol Cell Biochem 166:169-175.
69. Suresh Babu P, Srinivasan K 1998 Amelioration of renal lesions associated with diabetes by dietary curcumin in streptozotocin diabetic rats. Mol Cell Biochem 181:87-96.
70. Sajithlal G B, Chithra P, Chandrakasan G 1998 Effect of curcumin on the advanced glycation and cross-linking of collagen in diabetic rats. Biochem Pharmacol 56:1607-1614.
71. Nishizono S, Hayami T, Ikeda I, Imaizumi K 2000 Protection against the diabetogenic effect of feeding tert-butylhydroquinone to rats prior to the administration of streptozotocin. Biosci Biotechnol Biochem 64:1153-1158.
72. Kumar P A, Haseeb A, Suryanarayana P, Ehtesham N Z, Reddy G B 2005 Elevated expression of alphaA- and alphaB-crystallins in streptozotocin-induced diabetic rat. Arch Biochem Biophys 444:77-83.
73. Majithiya J B, Balaraman R 2005 Time-dependent changes in antioxidant enzymes and vascular reactivity of aorta in streptozotocin-induced diabetic rats treated with curcumin. J Cardiovasc Pharmacol 46:697-705.
74. Mahesh T, Balasubashini M S, Menon V P 2005 Effect of photo-irradiated curcumin treatment against oxidative stress in streptozotocin-induced diabetic rats. J Med Food 8:251-255.
75. Majithiya J B, Balaraman R, Giridhar R, Yadav M R 2005 Effect of bis(curcumino)oxovanadium complex on non-diabetic and streptozotocin-induced diabetic rats. J Trace Elem Med Biol 18:211-217.
76. Suryanarayana P, Saraswat M, Mrudula T, Krishna T P, Krishnaswamy K, Reddy G B 2005 Curcumin and turmeric delay streptozotocin-induced diabetic cataract in rats. Invest Opthalmol V is Sci 46:2092-2099.
77. Mahesh T, Sri Balasubashini M M, Menon V P 2004 Photo-irradiated curcumin supplementation in streptozotocin-induced diabetic rats: effect on lipid peroxidation. Therapie 59:639-644.
78. Arun N, Nalini N 2002 Efficacy of turmeric on blood sugar and polyol pathway in diabetic albino rats. Plant Foods Hum Nutr 57:41-52.
79. Sidhu G S, Mani H, Gaddipati J P, Singh A K, Seth P, Banaudha K K, Patnaik G K, Maheshwari R K 1999 Curcumin enhances wound healing in streptozotocin induced diabetic rats and genetically diabetic mice. Wound Repair Regen 7:362-374.
80. Srivivasan A, Menon V P, Periaswamy V, Rajasekaran K N 2003 Protection of pancreatic beta-cell by the potential antioxidant bis-o-hydroxycinnamoyl methane, analogue of natural curcuminoid in experimental diabetes. J Pharm Pharm Sci 6:327-333.
81. Kumar P A, Suryanarayana P, Reddy P Y, Reddy G B 2005 Modulation of alpha-crystalline chaperone activity in diabetic rat lens by curcumin. Mol Vis 11:561-568.
82. Sharma S, Kulkarni S K, Agrewala J N, Chopra K 2006 Curcumin attenuates thermal hyperalgesia in a diabetic mouse model of neuropathic pain. Eur Pharmacol 536:256-261.
83. Yang H, Chen D, Cui Q C, Yuan X, Dou Q P 2006 Celastrol, a triterpene extracted from the Chinese “Thunder of God Vine” is a potent proteasome inhibitor and suppresses human prostate cancer growth in nude mice. Cancer Res 66(9):4758-4765.
84. Meng L, Mohan R, Kwok B H B, Elofsson M, Sin N, and Crews C M 1999 Epoxomicin, a potent and selective proteasome inhibitor, exhibits in vivo antiinflammatory activity. Proc. Natl. Acad. Sci. USA 96:10403-10408.
85. Sin N, Kim K B, Elofsson M, Meng L, Auth H, Kwok B H B, Crews, C M 1999 Total Synthesis Of The Potent Proteasome Inhibitor Epoxomicin: A Useful Tool For Understanding Proteasome Biology.
86. Stiles B L, Kuralwalla-Martinez C, Guo W, Gregorian C, Wang Y, Tian J, Magnuson M A, Wu H 2006 Selective deletion of Pten in pancreatic beta cells leads to increased islet mass and resistance to STZ-induced diabetes. Mol Cell Biol 26:2772-2781.
87. Barbosa H, Bordin S, Stoppiglia L, Silva K, Borelli M, Del Zotto H, Gagliardino J, Boschero A 2006 Islet Neogenesis Associated Protein (INGAP) modulates gene expression in cultured neonatal rat islets. Regul Pept 136:78-84.

The scope of the present invention is not limited by the description, examples, and suggested uses herein and modifications can be made without departing from the spirit of the invention. Thus, it is intended that the present invention cover modifications and variations of this invention provided that they come within the scope of the appended claims and their equivalents.

1. A method for treating or preventing diabetes comprising administering to a mammal an effective amount of a selective proteasome inhibitor to treat or prevent diabetes.

2. A method for treating or preventing type-2 diabetes mellitus comprising administering to a mammal an effective amount of a selective proteasome inhibitor to treat or prevent type-2 diabetes mellitus.

3. A method of modulating chronic low-grade inflammation comprising administering to a mammal in need thereof an effective amount of a selective proteasome inhibitor to modulate chronic low-grade inflammation.

4. The method according to claim 1, wherein the selective proteasome inhibitor is selected from the group consisting of inhibitors of proteasome caspase-like activity, inhibitors of proteasome trypsin-like activity, inhibitors of proteasome chymotrypsin-like activity, inhibitors of all proteasome activities, and combinations thereof.

5. The method according to claim 4, wherein the inhibitors of proteasome caspase-like activity are selected from the group consisting of Ac-Ala-Pro-Nle-Asp-H, YU102, Calpain Inhibitor I (ALLN), ALLM (Calpain Inhibitor), Z-Ile-Glu(OBut)-Ala-Leu-H(PSI), MG115 (Z-Leu-Leu-Nva-H), MG-132 (Z-Leu-Leu-Leu-H), MG-262 (Z-Leu-Leu-Leu-B(OH)2), Z-(Leu)3-vinyl sulfone, Z-Pro-Nle-Asp-H, and combinations thereof.

6. The method according to claim 4, wherein the inhibitors of proteasome trypsin-like activity are selected from the group consisting of lactacystin, clasto-lactacystin β-lactone, NIP-(Leu)3-vinyl sulfone, TLCK, and combinations thereof.

7. The method according to claim 4, wherein the inhibitors of proteasome chymotrypsin-like activity are selected from the group consisting of aclacinomycin A (Aclarubicin), calpain inhibitor I (ALLN), ALLM (Calpain Inhibitor), epigallocatechin gallate, epoxomicin, gliotoxin, lactacystin, clasto-lactacystin β-lactone, NIP-(Leu)3-vinyl sulfone, phepropeptin A, phepropeptin B, phepropeptin C, Phepropeptin D, phepropeptin A, B, C, D Inhibitor Pack, TPCK, Z-Ile-Glu(OBut)-Ala-Leu-H(PSI), Z-(Leu)3-vinyl sulfone, MG115 (Z-Leu-Leu-Nva-H), MG-132 (Z-Leu-Leu-Leu-H), MG-262 (Z-Leu-Leu-Leu-B(OH)₂), Z-Leu-Leu-Tyr-COCHO, and combinations thereof.

8. The method according to claim 4, wherein the inhibitors of all proteasome activities are selected from the group consisting of ada-(Ahx)3-(Leu)3-vinyl sulfone, ada-Lys(biotinyl)-(Ahx)3-(Leu)3-vinyl sulfone, ada-Tyr-(Ahx)3-(Leu)3-vinyl sulfone, bactenecin 5 precursor peptide (Bac5-GRR), PR11, PR26, PR39, and combinations thereof.

9. The method according to claim 1, wherein the selective proteasome inhibitor is selected from the group consisting of ubiquitin+1 (Ub+1), ubiquitin5+1 (Ub5+1), and combinations thereof.

10. The method according to claim 1, wherein the selective proteasome inhibitor is selected from the group consisting of curcumin, epoxomicin, celastrol, derivatives thereof, and combinations thereof.

11. The method according to claim 10, wherein the selective proteasome inhibitor is curcumin.

12. The method according to claim 10, wherein the selective proteasome inhibitor is epoxomicin.

13. The method according to claim 10, wherein the selective proteasome inhibitor is celastrol.

14. The method according to claim 1, wherein the diabetes is type-2 diabetes mellitus.

15. The method according to claim 1, wherein the mammal is a human.

16. The method according to claim 1, wherein the effective amount is about 1 mg/kg/day to about 150 mg/kg/day.

17. The method according to claim 16, wherein the effective amount is about 50 mg/kg/day to about 150 mg/kg/day.

18. The method according to claim 1, wherein the effective amount is about 1.0 g/day to about 18 g/day.

19. The method according to claim 18, wherein the effective amount is about 1 g/day to about 1.5 gram per day.

20-50. (canceled)

51. A unit dosage form for treating or preventing type-2 diabetes mellitus comprising an effective amount of a selective proteasome inhibitor to treat or prevent type-2 diabetes mellitus in a mammal.


SEQUENCE LISTING (9 sequences)

SEQ ID NO: 1 - protein, 4 aa, Artificial sequence
  Synthetic construct
APXD

SEQ ID NO: 2 - protein, 4 aa, Artificial sequence
  Proteasome inhibitor
IEAL

SEQ ID NO: 3 - protein, 4 aa, Artificial sequence
  Proteasome inhibitor
XLLL

SEQ ID NO: 4 - protein, 6 aa, Artificial sequence
  Proteasome inhibitor
XXXLLL

SEQ ID NO: 5 - protein, 7 aa, Artificial sequence
  Proteasome inhibitor
KXXXLLL

SEQ ID NO: 6 - protein, 7 aa, Artificial sequence
  Proteasome inhibitor
YXXXLLL

SEQ ID NO: 7 - protein, 46 aa, Artificial sequence
  Proteasome inhibitor
RFRPPIRRPPIRPPFYPPFRPPIRPPIFPPIRPPFRPPLGPFPGRR

SEQ ID NO: 8 - protein, 11 aa, Artificial sequence
  Proteasome inhibitor
RRRPRPPYLPR

SEQ ID NO: 9 - protein, 26 aa, Artificial sequence
  Proteasome inhibitor
RRRPRPPYLPRPRPPPFFPPRLPPRI